(19) United States
(12) Patent Application Publication
(10) Pub. No.: US 2013/0040331 A1
(43) Pub. Date: Feb. 14, 2013

(54) LABEL-FREE METHODS RELATED TO hERG POTASSIUM ION CHANNELS

(71) Applicants: Ye Fang, Painted Post, NY (US); Haiyan Sun, Chandler, AZ (US)

(72) Inventors: Ye Fang, Painted Post, NY (US); Haiyan Sun, Chandler, AZ (US)

(73) Assignee: CORNING INCORPORATED, Corning, NY (US)

(21) Appl. No.: 13/519,246

(22) PCT Filed: Dec. 16, 2010
(86) PCT No.: PCT/US2010/060688

§ 371 Date: Jun. 26, 2012

Related U.S. Application Data
(60) Provisional application No. 61/291,731, filed on Dec. 31, 2009.

(51) Int. Cl. C12Q 1/02; G01N 27/26

(52) U.S. Cl. 435/29

(57) ABSTRACT

Disclosed are methods to classify human ether-à-go-go related gene (hERG) ion channel modulators using label-free biosensors. Disclosed is the use of label-free resonant waveguide grating (RWG) biosensors to reveal the patterns of the DMR signals of hERG modulators across three types of cell lines (a native cell line endogenously expressing hERG, a native cell line without hERG and its engineered cell line stably expressed hERG), as well as the corresponding modulation index of the modulators molecules against a hERG activator acting on a panel of markers/cells, particularly the known hERG activator mallotoxin DMR signals in the two hERG expressing cell lines.

### CROSS-REFERENCE TO RELATED APPLICATIONS

This application claims the benefit of priority under 35 U.S.C. §119(e) of U.S. Provisional Application Ser. No. 61/291,731 filed on Dec. 31, 2009.

### BACKGROUND

The human ether-à-go-go related gene (hERG) encodes the pore-forming a subunit of a voltage gated potassium channel (Kv11.1). hERG channel is expressed in various tissues including cardiac myocytes, neurons, pancreatic 13 cells, smooth muscles, and some cancer cells.

hERG current is best known as the major component of the delayed rectifier current Iₖᵣ in the heart which is important for the action potential repolarization. Genetic mutations in hERG channel have been known to cause the inherited long QT syndrome (LQT); a disease could result in patient sudden death. Drugs that can block hERG current, or inhibit hERG channel protein trafficking could cause the acquired LQTs. To minimize the drug induced cardiac risk, all compounds under consideration for Investigational New Drug (ND) applications need to be tested for hERG interaction in compliance with GLP principles according to the ICH S7A and ICH S7B guidelines. Conversely, mutations of hERG channel protein also were reported to cause long QT syndrome.

Disclosed are compositions and methods for identifying the action of molecules on hERG channels, as well as desirable molecules which activate or inhibit hERG channels.

### SUMMARY

Disclosed are methods to classify human ether-à-go-go related gene (hERG) ion channel modulators using label-free biosensors. Disclosed is the use of label-free resonant waveguide grating (RWG) biosensors to reveal the patterns of the DMR signals of hERG modulators across three types of cell lines (a native cell line endogenously expressing hERG, a native cell line without hERG and its engineered cell line stably expressed hERG), as well as the corresponding modulation index of the modulators molecules against a hERG activator acting on a panel of markers/cells, particularly the known hERG activator mallotoxin DMR signals in the two hERG expressing cell lines.

### BRIEF DESCRIPTION OF THE FIGURES

FIG. 1A-1D shows a method to evaluate and classify hERG modulators with label-free RWG biosensor cellular assays. (A) The DMR signal of hERG activator mallotoxin in the colon cancerous cell line HT29; (B) The DMR signal of hERG activator mallotoxin in the engineered HEK293 cell line (HEK-hERG) stably expressing hERG; (C) The DMR signal of hERG activator mallotoxin in native HEK293 cells; (D) The modulation index of mallotoxin against the mallotoxin DMR signals in both HT29 and HEK-hERG cell lines. Mallotoxin was at 16 micromolar. In graphs A, B and C, the respective net-zero DMR signals of cells in response to the vehicle (i.e., buffer) only were included as negative controls. At lease two replicates were used to generate each averaged response. The same is for FIG. 2 to FIG. 5.

FIG. 2A-2D shows a method to evaluate and classify hERG modulators with label-free RWG biosensor cellular assays. (A) The DMR signal of an anti-inflammatory drug flufenamic acid in the colon cancer cell line HT29; (B) The DMR signal of flufenamic acid in the engineered HEK293 cell line (HEK-hERG) stably expressing hERG; (C) The DMR signal of flufenamic acid in native HEK293 cells; (D) The modulation index of flufenamic acid against the mallotoxin DMR signals in both HT29 and HEK-hERG cell lines. Flufenamic acid was assayed at 10 micromolar in all cells, while mallotoxin was at 16 micromolar. In graphs A, B and C, the respective net-zero DMR signals of cells in response to the vehicle (i.e., buffer) only were included as negative controls.

FIG. 3A-3D shows a method to evaluate and classify hERG modulators with label-free RWG biosensor cellular assays. (A) The DMR signal of an prostaglandin synthetase inhibitor diflunisal in the colon cancer cell line HT29; (B) The DMR signal of diflunisal in the engineered HEK293 cell line (HEK-hERG) stably expressing hERG; (C) The DMR signal of diflunisal in native HEK293 cells; (D) The modulation index of diflunisal against the mallotoxin DMR signals in both HT29 and HEK-hERG cell lines. Diflunisal was assayed at 10 micromolar in all cells, while mallotoxin was at 16 micromolar. In graphs A, B and C, the respective net-zero DMR signals of cells in response to the vehicle (i.e., buffer) only were included as negative controls.

FIG. 4A-4D shows a method to evaluate and classify hERG modulators with label-free RWG biosensor cellular assays. (A) The DMR signal of an ERK2 inhibitor AG-126 in the colon cancer cell line HT29; (B) The DMR signal of AG-126 in the engineered HEK293 cell line (HEK-hERG) stably expressing hERG; (C) The DMR signal of AG-126 in native HEK293 cells; (D) The modulation index of AG-126 against the mallotoxin DMR signals in both HT29 and HEK-hERG cell lines. AG-126 was assayed at 10 micromolar in all cells, while mallotoxin was at 16 micromolar. In graphs A, B and C, the respective net-zero DMR signals of cells in response to the vehicle (i.e., buffer) only were included as negative controls.

FIG. 5A-5D shows a method to evaluate and classify hERG modulators with label-free RWG biosensor cellular assays. (A) The DMR signal of an a potent EGF receptor inhibitor tyrphostin 51 in the colon cancer cell line HT29; (B) The DMR signal of tyrphostin 51 in the engineered HEK293 cell line (HEK-hERG) stably expressing hERG; (C) The DMR signal of tyrphostin 51 in native HEK293 cells; (D) The modulation index of tyrphostin 51 against the mallotoxin DMR signals in both HT29 and HEK-hERG cell lines. Tyrphostin 51 was assayed at 10 micromolar in all cells, while mallotoxin was at 16 micromolar. In graphs A, B and C, the respective net-zero DMR signals of cells in response to the vehicle (i.e., buffer) only were included as negative controls.

FIG. 6A-6B shows Rb⁺ flux measurements of hERG modulators using HEK-hERG cells under two different concentrations of KCl. (A) 5 mM KCl; (B) 40 mM KCl. The modulators were assayed at either 10 micromolar or 50 micromolar, when KCl was maintained at 5 mM. The modulators were assayed at 10 micromolar only, when KCl was maintained at 40 mM.

FIG. 7A-7B Dose dependent DMR signals of mallotoxin in the HEK-hERG cells. (A) Real time kinetic responses. (B) The mallotoxin DMR signals, calculated based on its amplitudes at 18 min after stimulation, were plotted as a function of mallotoxin doses.

FIG. 8A-8B (A) The DMR signals of the HEK-hERG cells in response to 16 μM mallotoxin in the absence (Control) and presence of three different hERG blockers, Cisapride, astemizole, and sertindole, respectively. Each compound was at 30 μM, and used to pretreat the cells for 30 min. (B) The dose-dependent inhibition of the mallotoxin DMR signals by astemizole.

FIG. 9 The mallotoxin DMR signals in HEK-hERG cells were robust. The averaged cellular DMR response induced by 16 micromolar mallotoxin was compared with the negative control (“Buffer”). 32 replicates were used to calculate the averaged responses.

FIG. 10A-10B The electrophysiological diagrams showing the effect of hERG modulators on the current of the HEK-hERG cells. (A) niflumic acid; and (B) flufenamic Acid. Each molecule was assayed at 50 micromolar. The gray curves represented the electrophysiological recording of HEK-hERG cells before the addition of a modulator, while the black curves showed the electrophysiological recording of the same cell after the addition of the corresponding modulator.

FIG. 11A-11B The electrophysiological diagrams showing the effect of hERG modulators on the current of the HEK-hERG cells. (A) Tyrphostin 51; and (B) Diflunisal. Each molecule was assayed at 50 micromolar. The gray curves represented the electrophysiological recording of HEK-hERG cells before the addition of a modulator, while the black curves showed the electrophysiological recording of the same cell after the addition of the corresponding modulator.

FIG. 12 The electrophysiological diagrams showing the effect of AG126 on the current of the HEK-hERG cells. AG126 was assayed at 50 micromolar. The gray curve represented the electrophysiological recording of HEK-hERG cells before the addition of AG126, while the black curves showed the electrophysiological recording of the same cell after the addition of AG126.

### DETAILED DESCRIPTION

### Compostions and Methods

hERG

The human ether-à-go-go related gene (hERG) product encodes for the pore-forming subunit of the rapid component of the delayed rectifier K+ channel that mediates repolarization of cardiac action potential. hERG is a voltage gated ion channel, and is involved in regulating the movement of potassium ions across the cell plasma membrane. In scientific literature, the hERG functions are largely discussed and described in the context of ion movement regulation and its consequence on cell biology. hERG ion channel is a large tetramer protein. Depending on cellular backgrounds it may be complexed with other proteins. Therefore the cell background can affect assays looking at hERG modulation. The disclosed assays use three types of cells and cell lines: a native hERG expressing cell line, a native cell line which does not express hERG, and an engineered cell line that is engineered to express hERG. Since label-free biosensor cellular assays rely on a generic readout, such as DMR signal using optical biosensor or impedance signal using electric biosensor, and the biosensor signal often contains systems cell biology information of a target of interest (e.g., hERG channel), there can be a high percentage of false positives that could be a result from screening using a single hERG expressing cell. Combining three types of cells for detecting hERG modulation using a label-free biosensor can not only significantly reduce false positives, but also can increase the quality of potential hERG modulators identified. Furthermore, by using the three cell lines, a high resolution assessment of hERG specific modulators is created. The disclosed assays also use a hERG activator, such as mallotoxin, to generate the modulation index of a molecule against the hERG activator induced DMR signals in both hERG expressing cell lines. Such modulation indexes can be used further classifying the mode of actions of molecules acting on hERG channel or hERG channel signaling complexes.

Besides playing a critical role in cardiac myocytes, increasing evidence has shown that hERG channel expression level was elevated in several types of cancer cells including leukemia, colon cancer, gastric cancer, breast cancer and lung cancer cells. It is not clear why the hERG channel is over expressed in cancer cells, but it is indicated that hERG channel plays a role in cancer cell proliferation.

hERG channel function is modulated by protein kinase A and protein kinase C involved pathways. hERG current is acutely inhibited when hERG protein is phosphorylated by the activation of cAMP dependent PKA. Elevated levels of cAMP and prolonged PKA activity can also increase the hERG protein expression. hERG current could also be modulated by adrenergic receptors through PKA and PKC.

hERG channel has unique pore region that can accommodate structure diverse channel blockers. A comparatively large inner cavity and the presence of particular aromatic amino acid residues (Y652 and F656) on the inner (S6) helices of the channel are important features that allow hERG to accommodate and bind disparate drugs.

In addition to the various hERG channel blockers, seven hERG channel activators have been identified, including RPR260243, NS1643, NS3623, PD-118057, PD-307243, mallotoxin and A-935142 (see Su, Z., et al. Electrophysiologic characterization of a novel hERG channel activator. Biochem Pharm 77:1383, 2009). These hERG activators have diverse chemical structures and enhance the hERG channel activity by different mechanisms. Among them, mallotoxin and A-935142 can shift the voltage dependent channel activation to less depolarized voltages. Electrophysiology studies showed that 10 micromolar MTX could shift the half maximal activation voltage (V1/2) to the hyperpolarizing direction for more than 25 mV. Among these known hERG activators, PD-118057, NS3623 and RPR260243 have been shown to shorten both the ventricular AP duration and the QT interval. RPR260243 and PD-118057 can reverse the AP prolonging effects of dofetilide. The mechanism of action of these channel activators is varied. NS1643 and NS3623 primarily reduce the inactivation of hERG by shifting its voltage dependence rightward; neither compound was designed to interact with the S5-pore linker and their sites of action with the hERG channel are as yet unknown. Mallotoxin strongly shifts the activation curve leftward, but also slows deactivation and has minor effects on inactivation.

hERG Assays

Because of the importance of hERG channels in drug safety issues, various technologies have been developed for hERG functional assays. Electrophysiology manual patch clamp technique has always been regarded as the gold standard for hERG studies. However, manual patch clamping requires experienced electrophysiologist and is very labor intensive. Recent years automated patch clamping technologies (such as IonWorks from Molecular Devices) have been developed which allows medium to high throughput testing of compound effect on hERG channel function. Different from patch clamping which measures hERG activity from a single ruptured cell, fluorescent membrane potential assays or Rb⁺ flux assays measure the hERG channel response from a population of cells. In addition, biochemical assays have also been used for measuring compound binding affinity with hERG protein. However, lacking physiologically functional assay readouts, particularly with high throughput, is considered a bottleneck in drug discovery for ion channels.

Since label-free biosensors, particularly optical biosensors, cellular assays are largely sensitive to mass redistribution within its sensing volume or detection volume, it is commonly believed that these biosensor cellular assays are not amenable to monitoring ion channel activities directly. However, since hERG is a quite large ion channel, and at least in many cancerous cells it could co-exist with several other signaling molecules including integrins and/or receptor tyrosine kinases to form a large signaling complex. There is evidence showing that hERG channel is involved in cell signaling. Furthermore, the recent discovery of several hERG activators, although via different mechanisms, can cause the activation of hERG channels at or near physiological conditions. Thus, we speculated that label-free RWG biosensor cellular assays can directly assay the activation and its subsequent signaling of hERG channels without applying voltages to the cells.

Label-free cellular assays are largely considered to be non-specific, due to its integrative and generic nature of the biosensor readout or output signals obtained. A given biosensor output signal induced by a compound could be originated from different possible cellular processes or signaling events. Also, the possibility of a compound having polypharmacology, which is quite common to almost all compounds, complicates the assignment of modes of action of the compound observed with these biosensor cellular assays. To address that, disclosed are methods to characterize, identify, and screen hERG modulators with much greater specificity and certainty.

Other proteins, such as other ion channels, such as the Toll-like receptors, can be screened and characterized in similar ways, according to the disclosed methods.

In certain label free cell assay methods, one has a cell line, a target, an activator (or modulator), and then a marker. These combinations can be used to assay for diverse arrays of modulators (See for example, WO2006108183 Fang, Y., et al. “Label-free biosensors and cells”).

Screening using a typical label free cell assay target approach (i.e., screening using a specific cell line expressing the target of interest, e.g., HEK-hERG cells) commonly leads to high false positives. This is because label-free cellular assays are often to profiling molecules at pathway level due to the integrative nature of the biosensor signal of the target receptor (meaning that modulators that not only act on the target of interest, but also on the target-associated pathway(s) are detected together from a screen). As a result, screening using the typical label-free cell assay target approach leads to information about the pathways and targets involved in these, but often exhibits low specificity at the target level. The methods disclosed herein use the information that can be gained from label free target assays and label free pathway assays, to achieve screen results with higher specificity at the target level.

The disclosed methods provide a higher resolution of assessment of modulators acting on a specific target then in previous label free integrated pharmacology methods, such as those disclosed in U.S. application Ser. No. 12/623,693. Fang, Y., Ferrie, A. M., Lahiri, J., and Tran, E. “Methods for Characterizing Molecules”, Filed Nov. 23, 2009 and U.S. application Ser. No. 12/623,708. Fang, Y., Ferrie, A. M., Lahiri, J., and Tran, E. “Methods of creating an index”, filed Nov. 23, 2009. U.S. application Ser. No. 12/623,693. Fang, Y., Ferrie, A. M., Lahiri, J., and Tran, E. “Methods for Characterizing Molecules”, Filed Nov. 23, 2009 and U.S. application Ser. No. 12/623,708. Fang, Y., Ferrie, A. M., Lahiri, J., and Tran, E. “Methods of creating an index”, filed Nov. 23, 2009, both of which are herein incorporated by reference at least for material related to label free assays and methods. In the methods disclosed in U.S. application Ser. No. 12/623,693. Fang, Y., Ferrie, A. M., Lahiri, J., and Tran, E. “Methods for Characterizing Molecules”, Filed Nov. 23, 2009 and U.S. application Ser. No. 12/623,708. Fang, Y., Ferrie, A. M., Lahiri, J., and Tran, E. “Methods of creating an index”, filed Nov. 23, 2009, a panel of markers is chosen and assayed, and this information provides information about the pathways in the cell connected to the markers. The disclosed methods use identified cells, based on appropriate pathways for specific targets. In certain embodiments, the information used from methods disclosed in U.S. application Ser. No. 12/623,693. Fang, Y., Ferrie, A. M., Lahiri, J., and Tran, E. “Methods for Characterizing Molecules”, Filed Nov. 23, 2009 and U.S. application Ser. No. 12/623,708. Fang, Y., Ferrie, A. M., Lahiri, J., and Tran, E. “Methods of creating an index”, filed Nov. 23, 2009, can be used to provide the information and identified cells which can be used in the methods disclosed herein.

Disclosed are molecules which have a heretofor unknown activity. Disclosed are over 3000 compounds, which have been tested in a hERG ion channel assay as well as in a label free biosensor assay. These compounds include BioMol 640 FDA approved drug library, BioMol 80 Kinase Inhibitor Library, BioMol ActiCom library, Corning Internal Reference Compound Library, and Corning Internal Compound Library. According to the disclosed methods, a subset of these compounds are identified as hERG modulators, which can classified into three classes: a hERG activator, a hERG inhibitor, and a hERG signaling activator that is capable of activating hERG signaling but with or without impact on hERG current.

The traditional hERG ion channel assay involves assaying ion flux such as Rb⁺ flux using ion absorption assays, or assaying hERG currents directly using patch clamping methods. Traditionally, a molecule which causes increase in Rb⁺ flux and/or hERG currents is referred to a hERG activator, while a molecule which inhibits Rb⁺ flux and/or hERG currents is referred to a hERG inhibitor. The disclosed methods have identified different classes of hERG activators, including hERG ion channel activators and hERG pathway activators. These hERG activators may or may not result in detectable biosensor signals in cells, using label-free biosensor cellular assays. A hERG activator that results in a detectable biosensor signal in a hERG expressing cell via hERG or hERG signaling complex is also referred to a label-free biosensor hERG activator. A hERG pathway activator is a molecule which cause cell signaling mediated via hERG or hERG-associated signaling complex in cells. These hERG pathway activators are also referred to hERG signaling activators. A hERG pathway activator can be a classical a hERG activator, or a hERG inhibitor, based on its ability to potentiate or inhibit hERG ion flux and/or hERG current, respectively.

The data herein discloses that there is a cell signaling activity of hERG, which can be dependent or independent on ion channel flux activity via hERG channels, heretofor unknown. A hERG pathway activator could lead to activation of specific pathway(s) downstream hERG channel directly, or hERG channel-associated signaling complex, thus triggering a detectable biosensor signal in cells. These pathways can include protein kinase A (PKA), protein kinase C (PKC), MAP kinase (MAPK) pathway, or integrin pathway, or any combinations of these pathways.

One clear indication from the present data is that prodrugs and drugs could effect hERG channels differently, as a traditional hERG activator and as a hERG pathway activator, respectively.

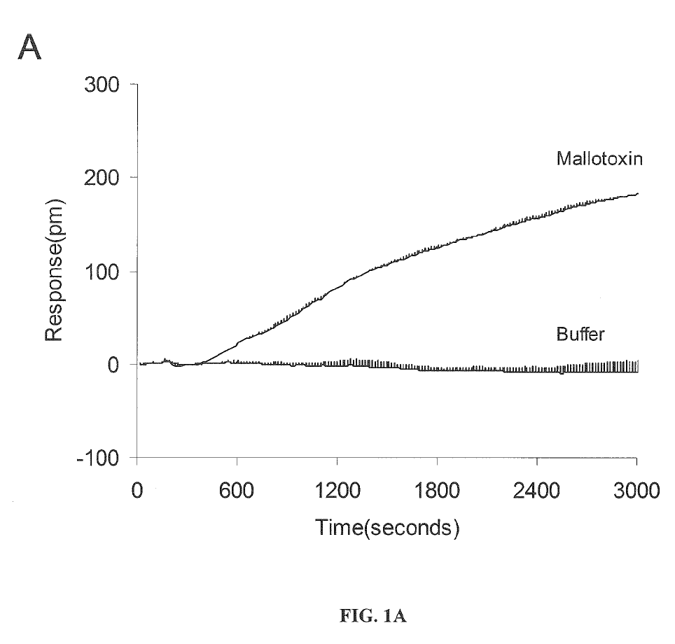

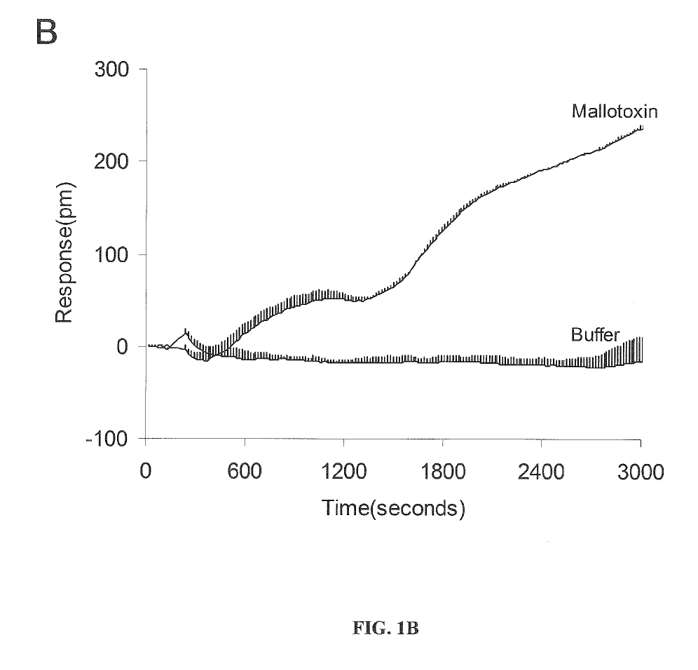

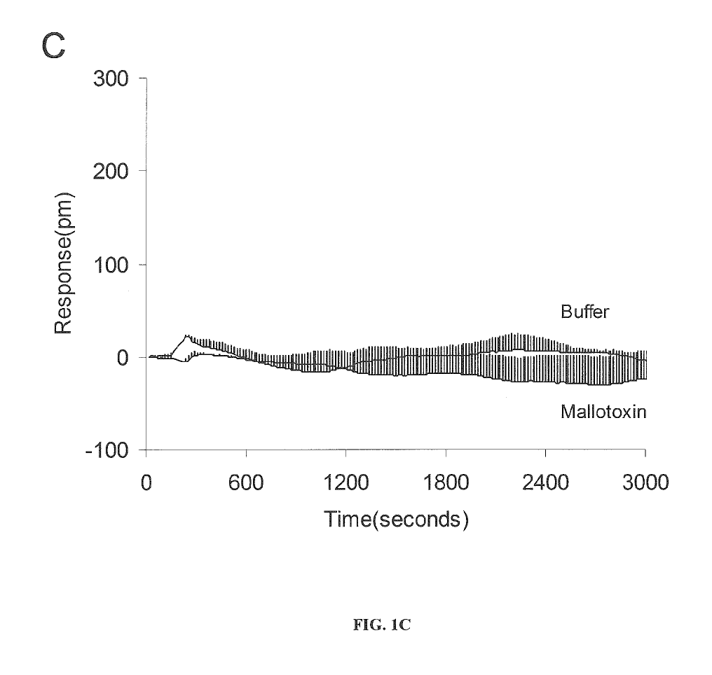

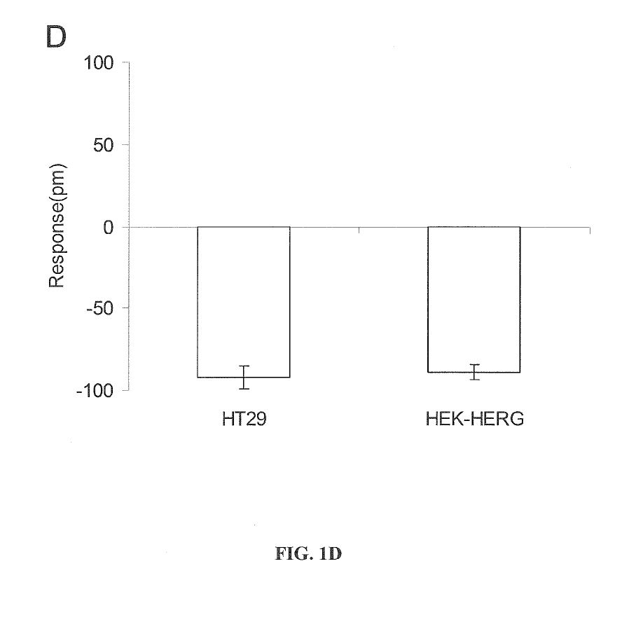

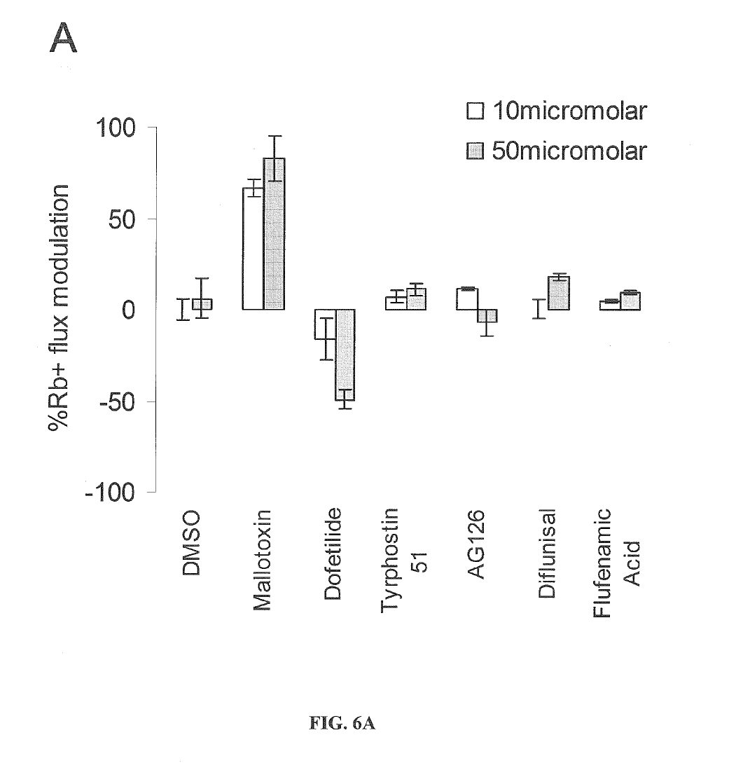

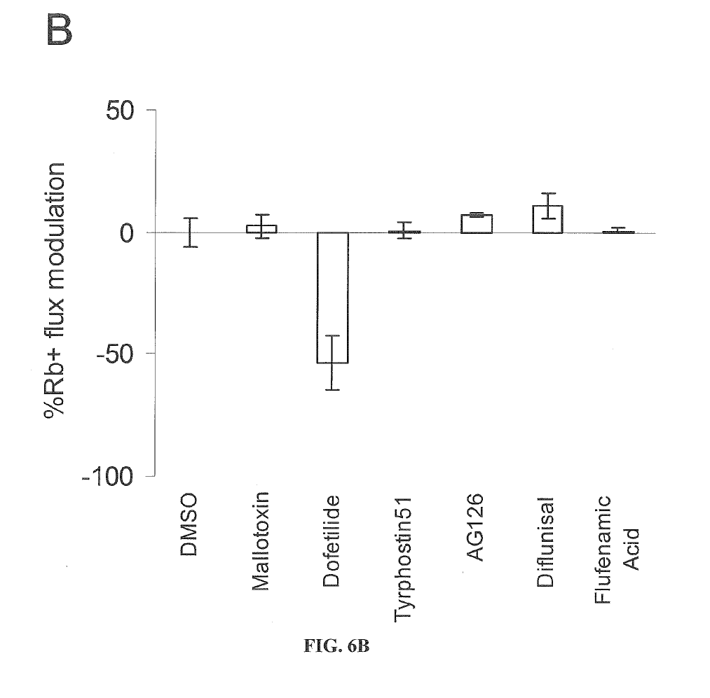

Mallotoxin is commercially available, and it is a label-free hERG pathway activator (FIG. 1), and also a strong hERG ion flux activator (FIG. 6) and a strong hERG current activator (data not shown).

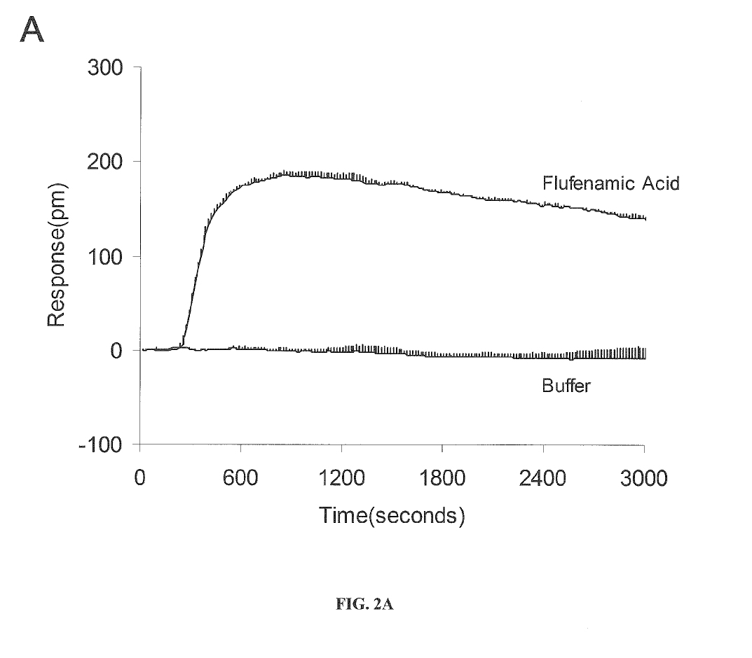

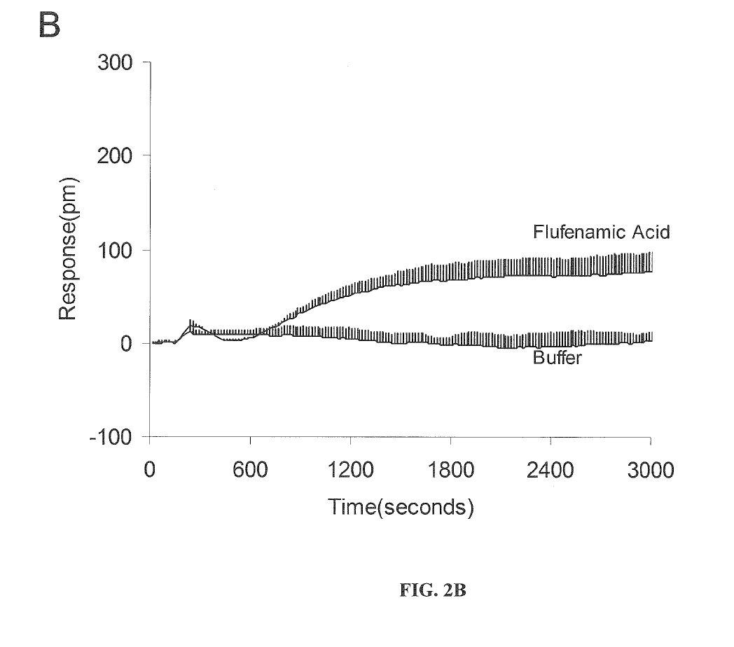

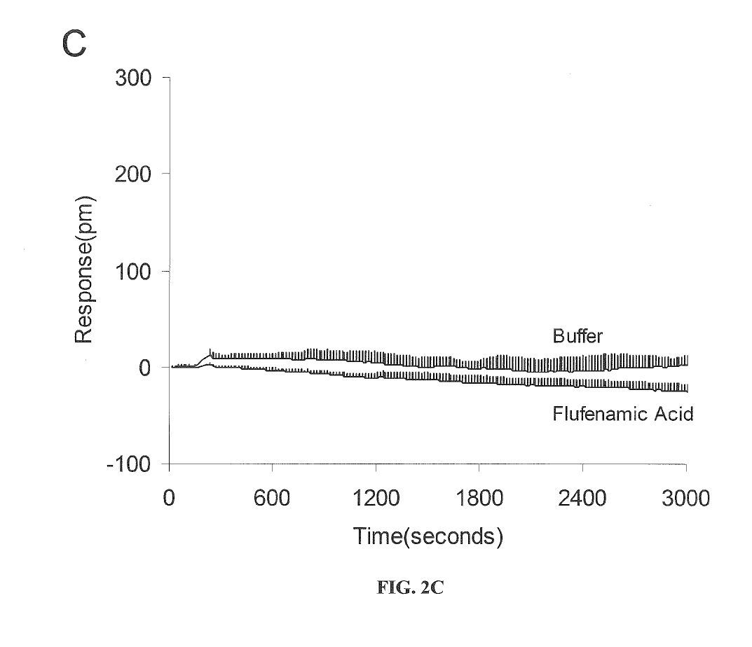

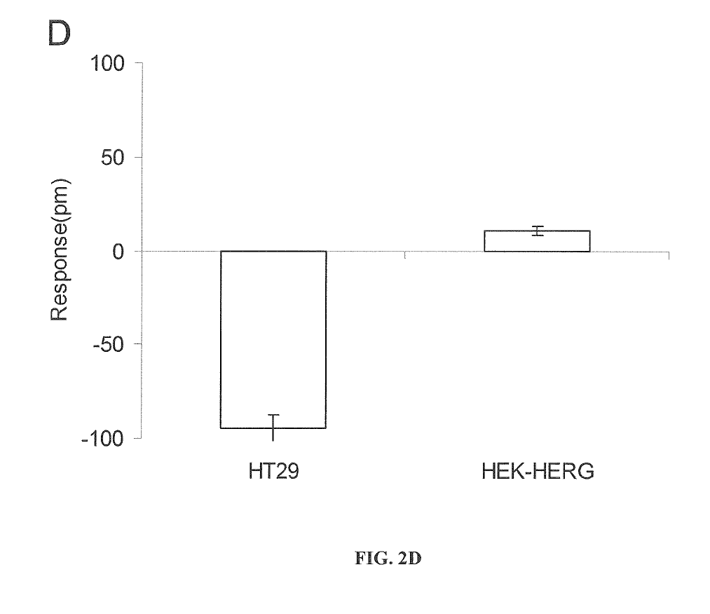

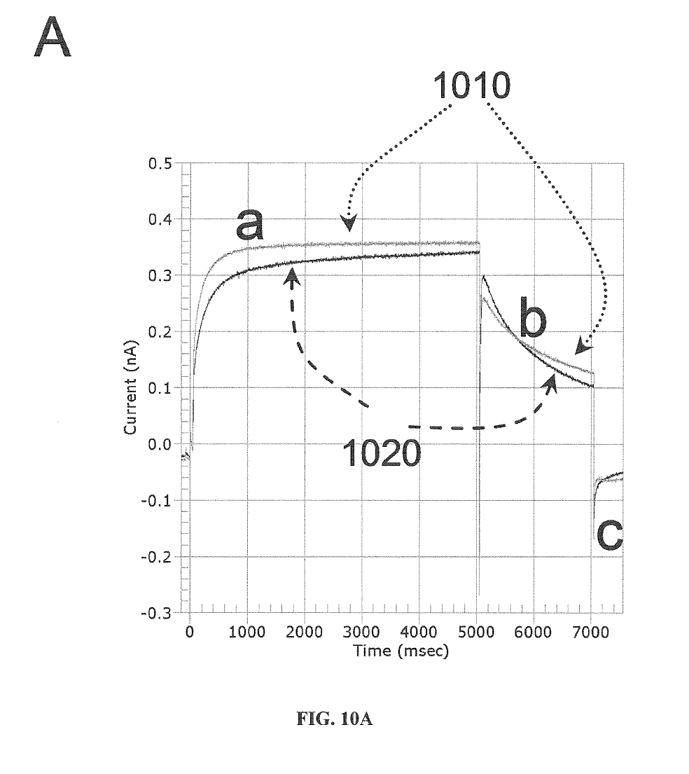

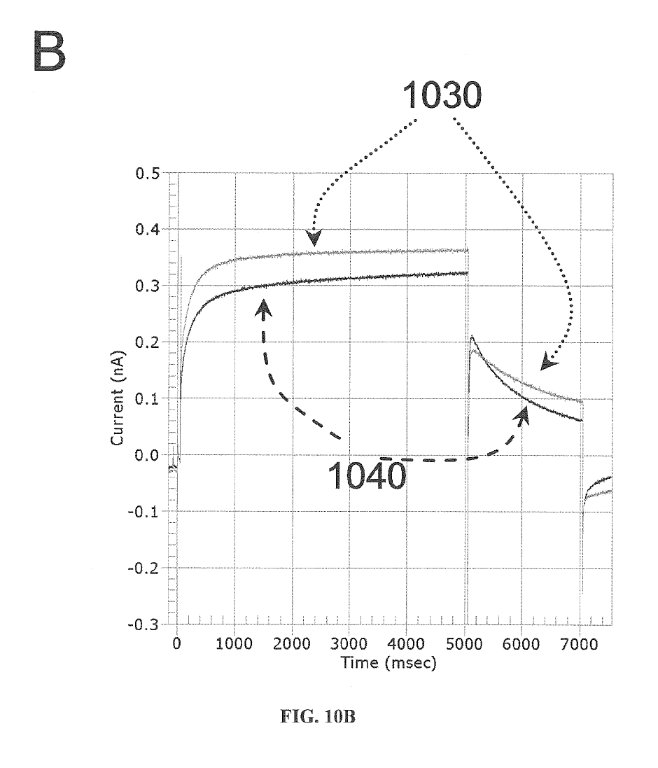

Also identified, flufenamic acid is a hERG pathway activator (FIG. 2), and is a weak hERG ion flux activator (FIG. 6), and a weak hERG current activator (FIG. 10B).

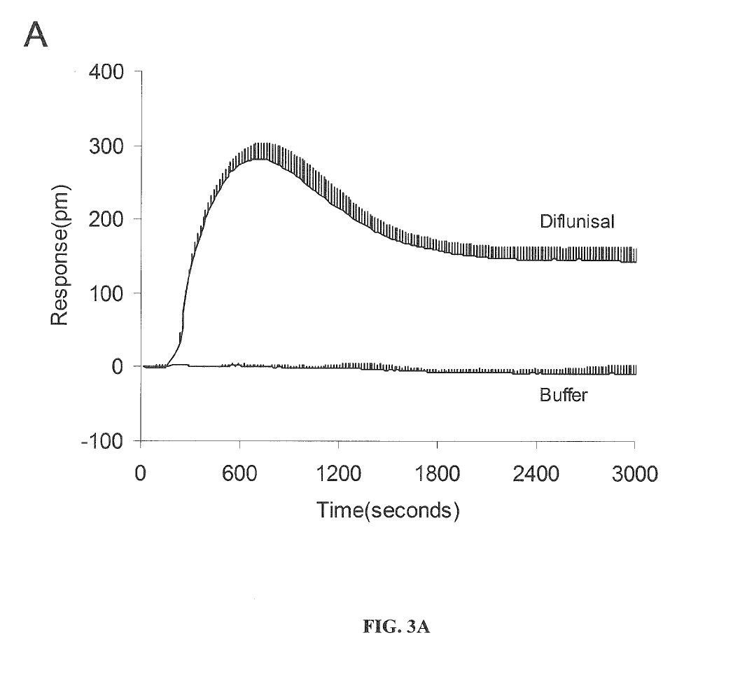

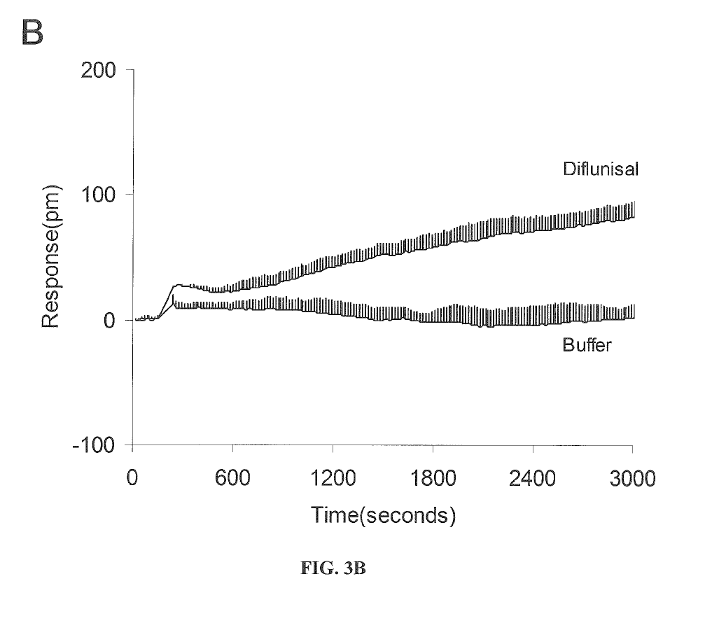

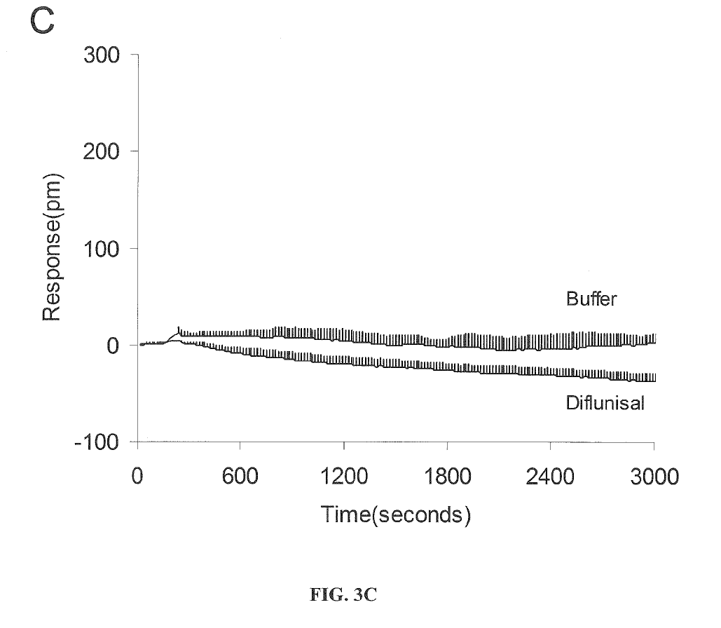

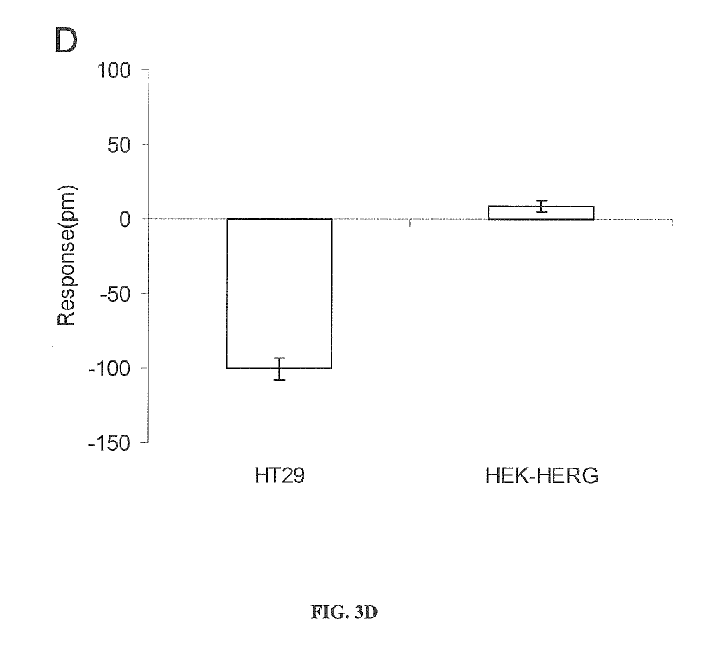

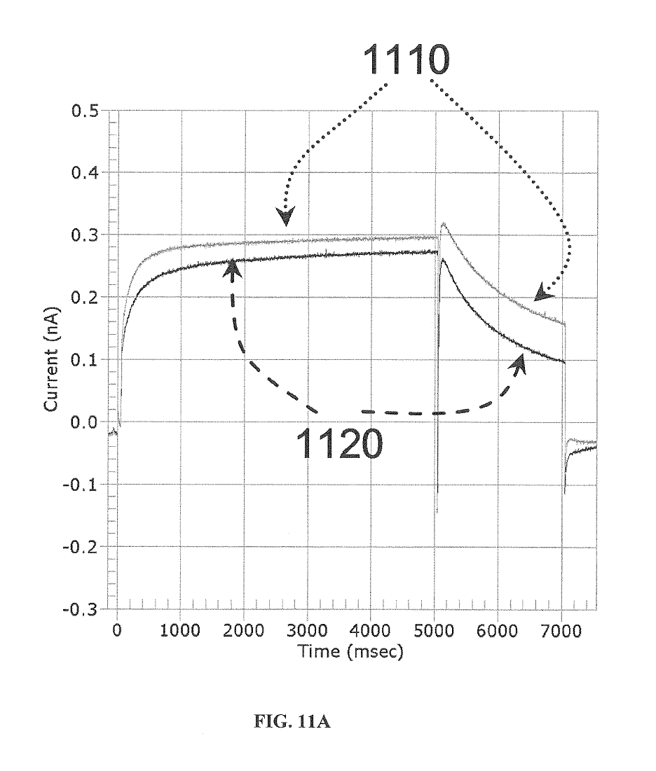

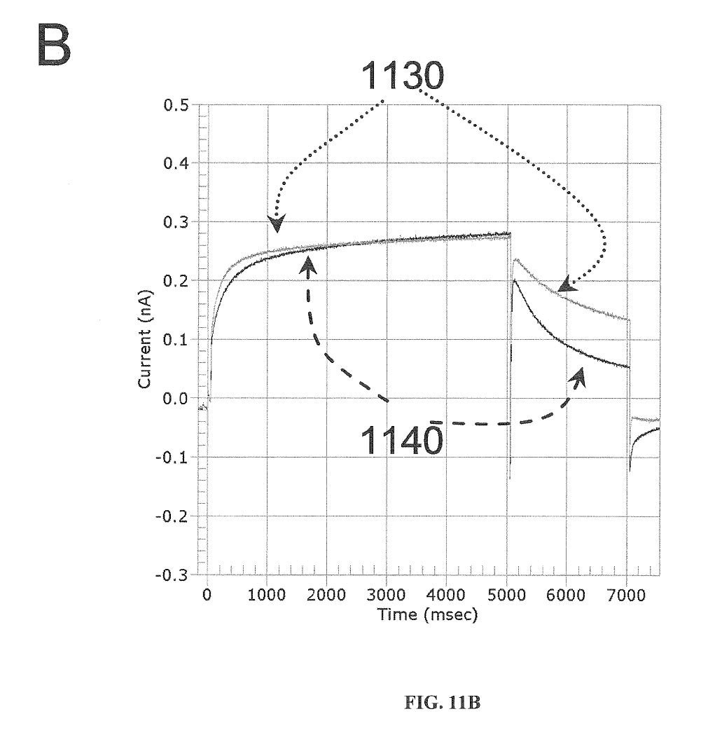

Also identified, difunisal is a hERG pathway activator (FIG. 3), and is a weak hERG ion flux activator (FIG. 6), and a hERG current inhibitor (FIG. 11B).

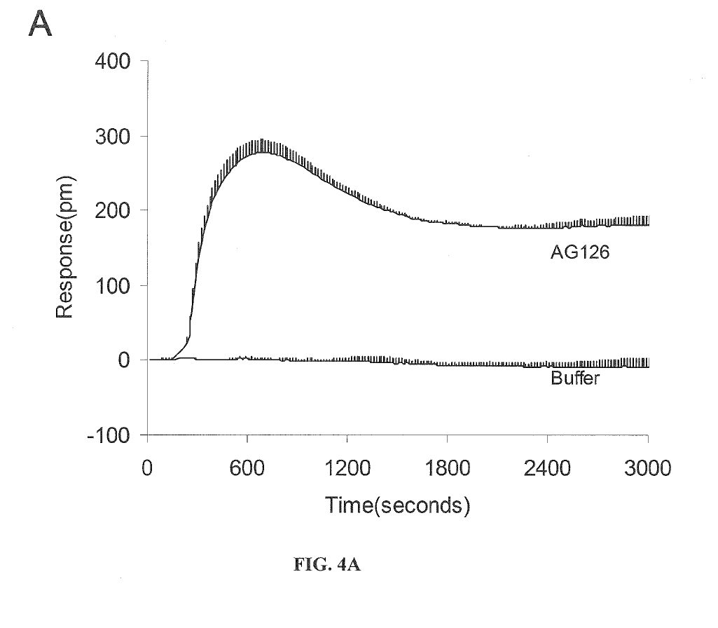

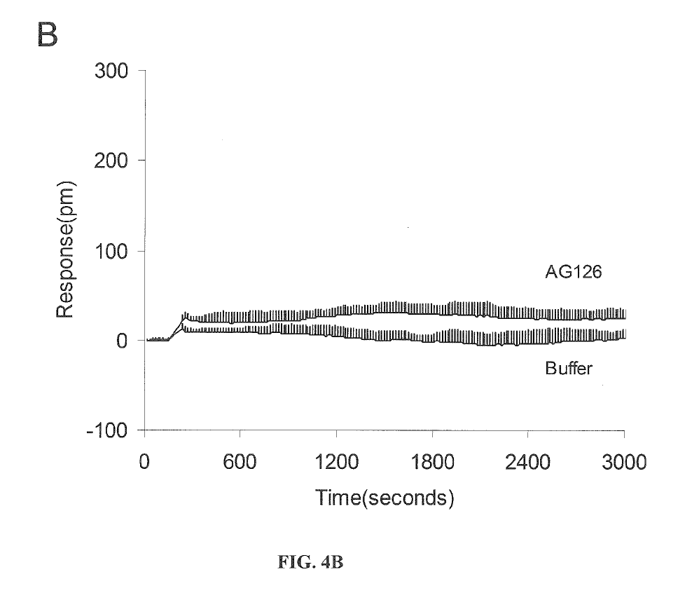

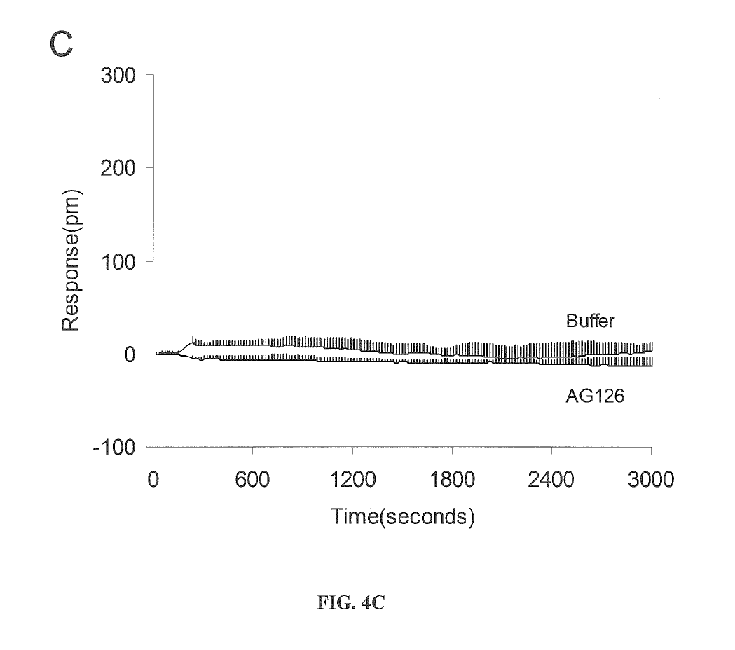

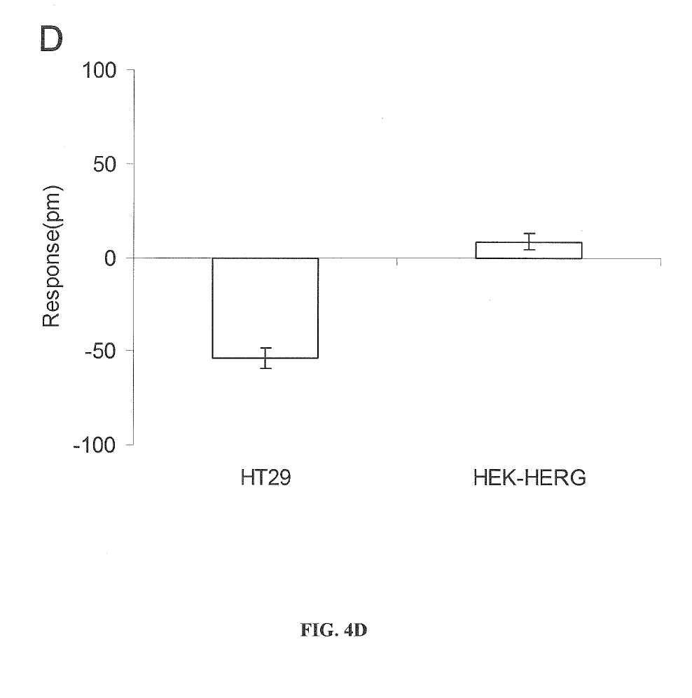

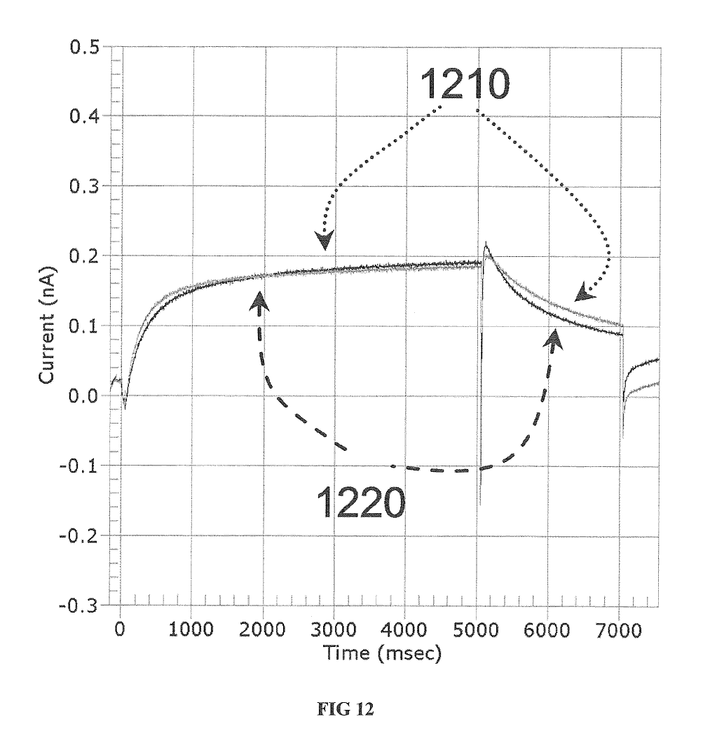

Also identified, AG126 is a hERG pathway activator, specifically in the endogenous hERG channel complex in HT29 (FIG. 4), and a non-effector in hERG ion flux (FIG. 6), and a non-effector in hERG current (FIG. 12).

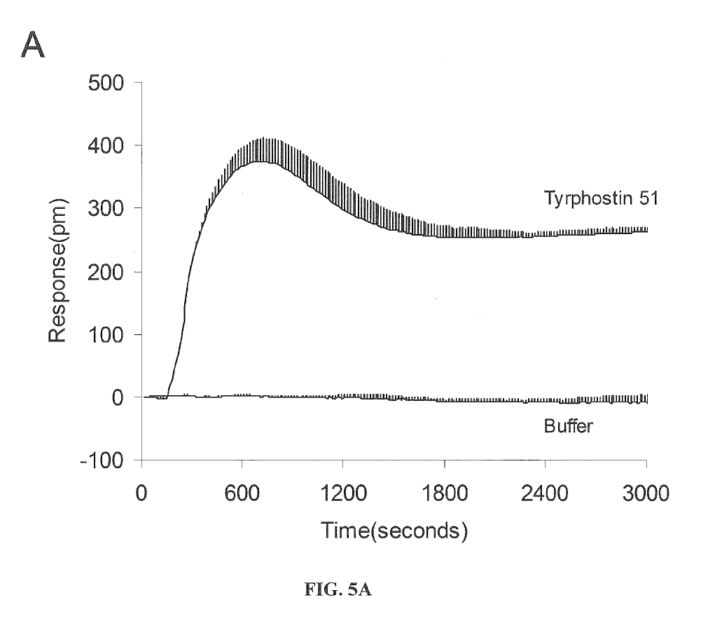

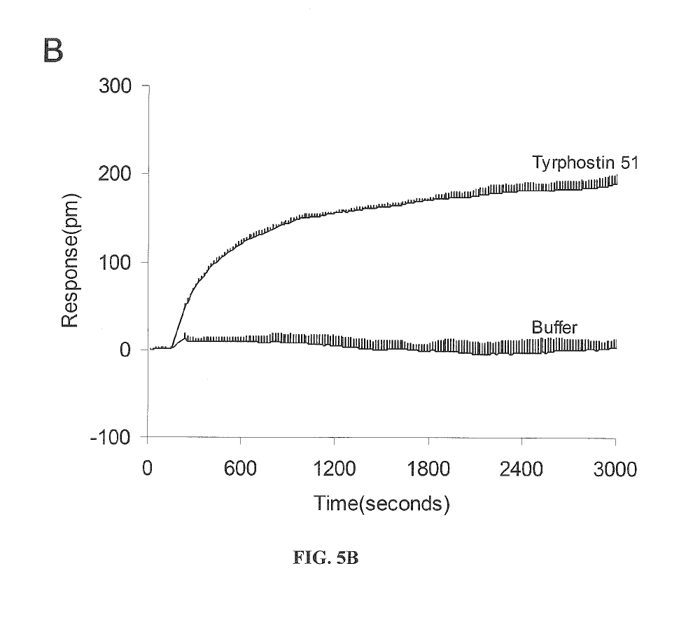

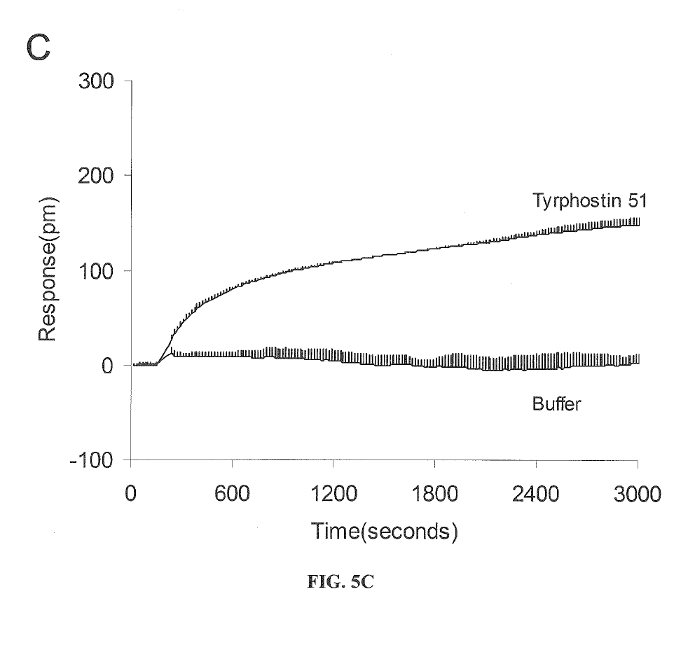

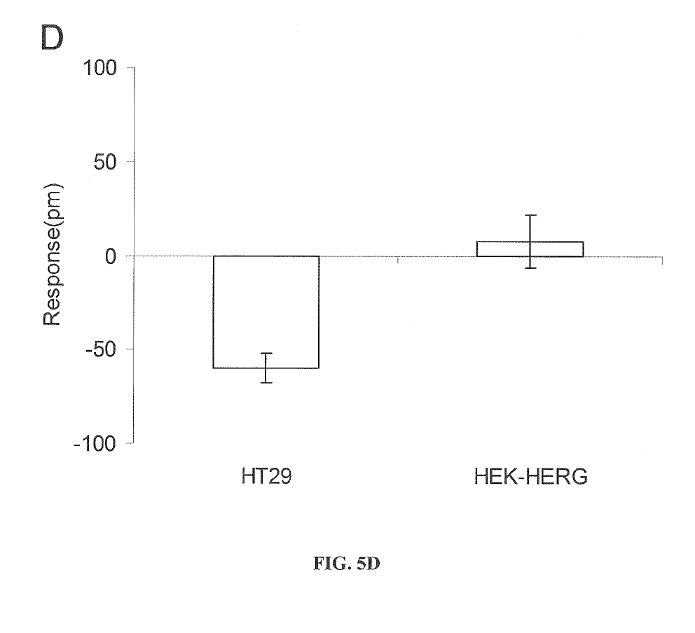

Also identified, tyrphostin 51 is a hERG pathway activator (FIG. 5), and a weak hERG ion flux activator (FIG. 6), and a hERG current inhibitor (FIG. 11A).

Also identified, niflumic acid is a hERG pathway activator (data not shown), and a hERG current activator (FIG. 10A).

Also identified, mallotoxin, RPR260243, NS1643, NS3623, PD-118057, PD-307243, A-935142, flufenamic acid, niflumic acid, and diflunisal are label-free biosensor hERG activators.

Disclosed are hERG modulators, hERG activator, label-free biosensor hERG activator, hERG pathway activator, hERG ion channel activator, hERG inhibitor, hERG pathway inhibitor, and hERG ion channel inhibitor. These classes and specific examples of each can be used, for example, in the methods disclosed herein.

The methods disclosed herein, as well as the compositions and compounds which can be used in the methods, can arise from a number of different classes, such as materials, substance, molecules, and ligands. Also disclosed is a specific subset of these classes, unique to label free biosensor assays, called markers, for example, mallotoxin as a marker for hERG activation.

It is understood that mixtures of these classes, such as a molecule mixture are also disclosed and can be used in the disclosed methods.

In certain methods, unknown molecules, test molecules, drug candidate molecules as well as known molecules can be used.

In certain methods or situations, modulating or modulators play a role. Likewise, known modulators can be used.

In certain methods, as well as compositions, cells are involved, and cells can undergo culturing and cell cultures can be used as discussed herein.

The methods disclosed herein involve assays that use biosensors. In certain assays, they are performed in either an agonism or antagonism mode. Often the assays involve treating cells with one or more classes, such as a material, a substance, or a molecule. It is also understood that subjects can be treated as well, as discuss herein.

In certain methods, contacting between a molecule, for example, and a cell can take place. In the disclosed methods, responses, such as cellular response, which can manifest as a biosensor response, such as a DMR response, can be detected. These and other responses can be assayed. In certain methods the signals from a biosensor can be robust biosensor signals or robust DMR signals.

The disclosed methods utilizing label free biosensors can produce profiles, such as primary profiles, secondary profiles, and modulation profiles. These profiles and others can be used for making determinations about molecules, for example, and can be used with any of the classes discussed herein.

Also disclosed are libraries and panels of compounds or compositions, such as molecules, cells, materials, or substances disclosed herein. Also disclosed are specific panels, such as marker panels and cell panels.

The disclosed methods can utilize a variety of aspects, such as biosensor signals, DMR signals, normalizing, controls, positive controls, modulation comparisons, indexes, biosensor indexes, DMR indexes, molecule biosensor indexes, molecule DMR indexes, molecule indexes, modulator biosensor indexes, modulator DMR indexes, molecule modulation indexes, known modulator biosensor indexes, known modulator DMR indexes, marker biosensor indexes, marker DMR indexes, modulating the biosensor signal of a marker, modulating the DMR signal, potentiating, and similarity of indexes.

Any of the compositions, compounds, or anything else disclosed herein can be characterized in any way disclosed herein.

Disclosed are methods that rely on characterizations, such as potentiate and inhibit and like words.

In certain methods, receptors or cellular targets are used. Certain methods can provide information about signaling pathway(s) as well as molecule-treated cells and other cellular processes.

In certain embodiments, a certain potency or efficacy becomes a characteristic, and the direct action (of a drug candidate molecule, for example) can be assayed.

The disclosed methods can be performed on or with samples.

Disclosed are methods to characterize modulators acting directly through hERG ion channel, or indirectly via hERG-associated signaling complexes using label-free biosensor cellular assays. Disclosed is the use of three types of cells: a cancerous cell line endogenously expressing hERG ion channels, a native cell line without endogenous hERG channels and its engineered cell line overexpressing hERG channels, for characterizing hERG modulators. Also disclosed is the use of mallotoxin or other hERG activator as a readout to further confirm the modes of action of hERG modulators. In addition, disclosed are methods that utilize additional assays, such as an ion flux assay, such as an Rb+ assay, membrane potential fluorescence assays, or patch clamping assays

Native cell lines endogenously expressing hERG channels include, but are not limited to, leukemia cell line HL60, gastric cancer cell line SGC7901 and MGC803, neuroblastoma cell line SH-SY5Y, mammary carcinoma cell line MCF-7, and human colon carcinoma cell HT-29, HCT8, and HCT116. Native cell lines without hERG include, but are not limited to, human embryonic kidney cell line HEK-293, and Chinese Ovary hamster cell line CHO—K1. Engineered cell lines overexpressing hERG include, but are not limited to, HEK-hERG and CHO-hERG cells. Cardiovascular or neuronal cells including primary cells having endogenous hERG channels can also be used.

hERG activators include, but are not limited to, mallotoxin, RPR260243, NS1643, NS3623, PD-118057, PD-307243, and A-935142.

Disclosed are methods of classifying a molecule for modulating hERG activity, comprising the steps: a. incubating a molecule individually with at least three different types of cells consisting of a native cell endogenously expressing hERG, an engineered cell stably expressing hERG and its parental cell without expressing hERG, and b. monitoring the molecule induced cellular response on each cell type with a label-free biosensor cellular assay,

Also disclosed are methods, further comprising as steps c) incubating a label-free biosensor hERG activator with each cell type in the presence of the molecule, d. monitoring the label-free biosensor hERG activator induced cellular response on each cell type in the presence of the molecule, e. generating a biosensor modulation index of the molecule against the label-free biosensor hERG activator induced biosensor signals in the two hERG expressing cell types.

Also disclosed are methods, wherein the label-free biosensor hERG activator is a hERG activator, hERG ion channel activator, or hERG pathway activator, wherein the hERG activator is selected from the group consisting of mallotoxin, RPR260243, NS1643, NS3623, PD-118057, PD-307243, A-935142, flufenamic acid, niflumic acid, or diflunisal, wherein the native hERG expressing cell line is selected from the group consisting of a leukemia cell line, a gastric cancer cell line, a neuroblastoma cell line, a mammary carcinoma cell line, and a human colon carcinoma cell line, a cardiovascular cell line, and a neuronal cell line, wherein the native hERG expressing cell line is selected from the group consisting of cell line HL60, cell line SGC7901, cell line MGC803, cell line SH-SY5Y, cell line MCF-7, cell line HT-29 HCT8, and cell line HCT116, wherein the native hERG non-expressing cell line is selected from the group consisting of a human embryonic kidney cell line or Chinese Ovary hamster cell line, wherein the native hERG non-expressing cell line is selected from the group consisting of cell line HEK-293 and cell line CHO—K1, wherein the hERG engineered cell line is selected from HEK-hERG or CHO-hERG, wherein the biosensor modulation index of the molecule is generated by normalizing the biosensor signal of the label-free biosensor hERG activator in the presence of the molecule to the biosensor signal of the label-free biosensor hERG activator in the absence of the molecule and/or with any method alone or in any combination.

Also disclosed are methods further comprising the step of comparing the effect of the molecule to the effect of a known modulator of hERG.

Disclosed are methods, wherein the hERG modulator is a hERG activator, a hERG ion channel activator, a hERG pathway activator, a hERG blocker, or a hERG pathway inhibitor, wherein the hERG modulator is selected from the group consisting of mallotoxin, RPR260243, NS1643, NS3623, PD-118057, PD-307243, and A-935142, AG-126, flufenamic acid, diflunisal, dofetilide, and tyrphostin 51, cisapride, astemizole, or sertindole and/or with any method alone or in any combination.

Also disclosed are methods, further comprising the step of determining if the molecule is a hERG modulator, further comprising identifying a hERG modulator when the molecule has a biosensor index similar to a hERG modulator, further comprising identifying a hERG activator when the molecule has a biosensor index similar to a known hERG activator, further comprising identifying a hERG inhibitor when the molecule has a biosensor index similar to a hERG inhibitor, further comprising assaying the molecule in a hERG ion flux assay test, and/or with any method alone or in any combination.

Also disclosed are methods, wherein the ion flux assay is a Rb⁺ ion flux assay, or a hERG current assay using electrophysiology patch clamping, wherein the molecule identified as a hERG activator in the label-free biosensor assay, regardless of its action in the hERG ion flux assay, is determined to be a hERG activator, wherein the molecule identified as an activator in the label free biosensor assay, but not identified as an activator in the hERG ion flux assay, is determined to be a hERG pathway activator, wherein the molecule identified as an activator in the label free biosensor assay, and also identified as an activator in the hERG ion flux assay, is determined to be a hERG ion channel activator, and/or with any method alone or in any combination.

### DEFINITIONS

Various embodiments of the disclosure will be described in detail with reference to drawings, if any. Reference to various embodiments does not limit the scope of the disclosure, which is limited only by the scope of the claims attached hereto. Additionally, any examples set forth in this specification are not intended to be limiting and merely set forth some of the many possible embodiments for the claimed invention.

A

As used in the specification and the appended claims, the singular forms “a,” “an” and “the” or like terms include plural referents unless the context clearly dictates otherwise. Thus, for example, reference to “a pharmaceutical carrier” includes mixtures of two or more such carriers, and the like.

Abbreviations

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

Assaying

Assaying, assay, or like terms refers to an analysis to determine a characteristic of a substance, such as a molecule or a cell, such as for example, the presence, absence, quantity, extent, kinetics, dynamics, or type of an a cell's optical or bioimpedance response upon stimulation with one or more exogenous stimuli, such as a ligand or marker. Producing a biosensor signal of a cell's response to a stimulus can be an assay.

Assaying the Response

“Assaying the response” or like terms means using a means to characterize the response. For example, if a molecule is brought into contact with a cell, a biosensor can be used to assay the response of the cell upon exposure to the molecule.

Agonism and Antagonism Mode

The agonism mode or like terms is the assay wherein the cells are exposed to a molecule to determine the ability of the molecule to trigger biosensor signals such as DMR signals, while the antagonism mode is the assay wherein the cells are exposed to a maker in the presence of a molecule to determine the ability of the molecule to modulate the biosensor signal of cells responding to the marker.

Biosensor

Biosensor or like terms refer to a device for the detection of an analyte that combines a biological component with a physicochemical detector component. The biosensor typically consists of three parts: a biological component or element (such as tissue, microorganism, pathogen, cells, or combinations thereof), a detector element (works in a physicochemical way such as optical, piezoelectric, electrochemical, thermometric, or magnetic), and a transducer associated with both components. The biological component or element can be, for example, a living cell, a pathogen, or combinations thereof. In embodiments, an optical biosensor can comprise an optical transducer for converting a molecular recognition or molecular stimulation event in a living cell, a pathogen, or combinations thereof into a quantifiable signal.

Biosensor Response

A “biosensor response”, “biosensor output signal”, “biosensor signal” or like terms is any reaction of a sensor system having a cell to a cellular response. A biosensor converts a cellular response to a quantifiable sensor response. A biosensor response is an optical response upon stimulation as measured by an optical biosensor such as RWG or SPR or it is a bioimpedence response of the cells upon stimulation as measured by an electric biosensor. Since a biosensor response is directly associated with the cellular response upon stimulation, the biosensor response and the cellular response can be used interchangeably, in embodiments of disclosure.

Biosensor Signal

A “biosensor signal” or like terms refers to the signal of cells measured with a biosensor that is produced by the response of a cell upon stimulation.

Cell

Cell or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

A cell can include different cell types, such as a cell associated with a specific disease, a type of cell from a specific origin, a type of cell associated with a specific target, or a type of cell associated with a specific physiological function. A cell can also be a native cell, an engineered cell, a transformed cell, an immortalized cell, a primary cell, an embryonic stem cell, an adult stem cell, a cancer stem cell, or a stem cell derived cell.

Human consists of about 210 known distinct cell types. The numbers of types of cells can almost unlimited, considering how the cells are prepared (e.g., engineered, transformed, immortalized, or freshly isolated from a human body) and where the cells are obtained (e.g., human bodies of different ages or different disease stages, etc).

Cell Culture

“Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also the culturing of complex tissues and organs.

Cell Panel

A “cell panel” or like terms is a panel which comprises at least two types of cells. The cells can be of any type or combination disclosed herein.

Cellular Response

A “cellular response” or like terms is any reaction by the cell to a stimulation.

Cellular Process

A cellular process or like terms is a process that takes place in or by a cell. Examples of cellular process include, but not limited to, proliferation, apoptosis, necrosis, differentiation, cell signal transduction, polarity change, migration, or transformation.

Cellular Target

A “cellular target” or like terms is a biopolymer such as a protein or nucleic acid whose activity can be modified by an external stimulus. Cellular targets are most commonly proteins such as enzymes, kinases, ion channels, and receptors.

Characterizing

Characterizing or like terms refers to gathering information about any property of a substance, such as a ligand, molecule, marker, or cell, such as obtaining a profile for the ligand, molecule, marker, or cell.

Comprise

Throughout the description and claims of this specification, the word “comprise” and variations of the word, such as “comprising” and “comprises,” means “including but not limited to,” and is not intended to exclude, for example, other additives, components, integers or steps.

Consisting Essentially of

“Consisting essentially of” in embodiments refers, for example, to a surface composition, a method of making or using a surface composition, formulation, or composition on the surface of the biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface modifier or condition, a particular ligand candidate, or like structure, material, or process variable selected. Items that may materially affect the basic properties of the components or steps of the disclosure or may impart undesirable characteristics to the present disclosure include, for example, decreased affinity of the cell for the biosensor surface, aberrant affinity of a stimulus for a cell surface receptor or for an intracellular receptor, anomalous or contrary cell activity in response to a ligand candidate or like stimulus, and like characteristics.

Components

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these molecules may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Contacting

Contacting or like terms means bringing into proximity such that a molecular interaction can take place, if a molecular interaction is possible between at least two things, such as molecules, cells, markers, at least a compound or composition, or at least two compositions, or any of these with an article(s) or with a machine. For example, contacting refers to bringing at least two compositions, molecules, articles, or things into contact, i.e. such that they are in proximity to mix or touch. For example, having a solution of composition A and cultured cell B and pouring solution of composition A over cultured cell B would be bringing solution of composition A in contact with cell culture B. Contacting a cell with a ligand would be bringing a ligand to the cell to ensure the cell have access to the ligand.

It is understood that anything disclosed herein can be brought into contact with anything else. For example, a cell can be brought into contact with a marker or a molecule, a biosensor, and so forth.

Compounds and Compositions

Compounds and compositions have their standard meaning in the art. It is understood that wherever, a particular designation, such as a molecule, substance, marker, cell, or reagent compositions comprising, consisting of, and consisting essentially of these designations are disclosed. Thus, where the particular designation marker is used, it is understood that also disclosed would be compositions comprising that marker, consisting of that marker, or consisting essentially of that marker. Where appropriate wherever a particular designation is made, it is understood that the compound of that designation is also disclosed. For example, if particular biological material, such as EGF, is disclosed EGF in its compound form is also disclosed.

Control

The terms control or “control levels” or “control cells” or like terms are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects or objects or reagents etc are treated as in a parallel experiment except for omission of the procedure or agent or variable etc under test and which is used as a standard of comparison in judging experimental effects. Thus, the control can be used to determine the effects related to the procedure or agent or variable etc. For example, if the effect of a test molecule on a cell was in question, one could a) simply record the characteristics of the cell in the presence of the molecule, b) perform a and then also record the effects of adding a control molecule with a known activity or lack of activity, or a control composition (e.g., the assay buffer solution (the vehicle)) and then compare effects of the test molecule to the control. In certain circumstances once a control is performed the control can be used as a standard, in which the control experiment does not have to be performed again and in other circumstances the control experiment should be run in parallel each time a comparison will be made.

Chemistry Terms

Alkyl

The term “alkyl” as used herein is a branched or unbranched saturated hydrocarbon moiety. “Unbranched” or “Branched” alkyls comprise a non-cyclic, saturated, straight or branched chain hydrocarbon moiety having from 1 to 24 carbons, 1 to 12 carbons, 1 to 6 carbons, or 1 to 4 carbon atoms. Examples of such alkyl radicals include methyl, ethyl, propyl, butyl, pentyl, hexyl, heptyl, octyl, n-propyl, iso-propyl, butyl, n-butyl, sec-butyl, t-butyl, amyl, t-amyl, n-pentyl and the like. Lower alkyls comprise a noncyclic, saturated, straight or branched chain hydrocarbon residue having from 1 to 4 carbon atoms, i.e., C₁-C₄ alkyl.

Moreover, the term “alkyl” as used throughout the specification and claims is intended to include both “unsubstituted alkyls” and “substituted alkyls”, the later denotes an alkyl radical analogous to the above definition that is further substituted with one, two, or more additional organic or inorganic substituent groups. Suitable substituent groups include but are not limited to H, alkyl, alkenyl, alkynyl, hydroxyl, cycloalkyl, heterocyclyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. It will be understood by those skilled in the art that an “alkoxy” can be a substituted of a carbonyl substituted “alkyl” forming an ester. When more than one substituent group is present then they can be the same or different. The organic substituent moieties can comprise from 1 to 12 carbon atoms, or from 1 to 6 carbon atoms, or from 1 to 4 carbon atoms. It will be understood by those skilled in the art that the moieties substituted on the “alkyl” chain can themselves be substituted, as described above, if appropriate.

Alkenyl

The term “alkenyl” as used herein is an alkyl residue as defined above that also comprises at least one carbon-carbon double bond in the backbone of the hydrocarbon chain. Examples include but are not limited to vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl and the like. The term “alkenyl” includes dienes and trienes of straight and branch chains.

Alkynyl

The term “alkynyl” as used herein is an alkyl residue as defined above that comprises at least one carbon-carbon triple bond in the backbone of the hydrocarbon chain. Examples include but are not limited ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

Cycloalkyl

The term “cycloalkyl” as used herein is a saturated hydrocarbon structure wherein the structure is closed to form at least one ring. Cycloalkyls typically comprise a cyclic radical containing 3 to 8 ring carbons, such as cyclopropyl, cyclobutyl, cyclopentyl, cyclopenyl, cyclohexyl, cycloheptyl and the like. Cycloalkyl radicals can be multicyclic and can contain a total of 3 to 18 carbons, or preferably 4 to 12 carbons, or 5 to 8 carbons. Examples of multicyclic cycloalkyls include decahydronapthyl, adamantyl, and like radicals.

Moreover, the term “cycloalkyl” as used throughout the specification and claims is intended to include both “unsubstituted cycloalkyls” and “substituted cycloalkyls”, the later denotes an cycloalkyl radical analogous to the above definition that is further substituted with one, two, or more additional organic or inorganic substituent groups that can include but are not limited to hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. When the cycloalkyl is substituted with more than one substituent group, they can be the same or different. The organic substituent groups can comprise from 1 to 12 carbon atoms, or from 1 to 6 carbon atoms, or from 1 to 4 carbon atoms.

Cycloalkenyl

The term “cycloalkenyl” as used herein is a cycloalkyl radical as defined above that comprises at least one carbon-carbon double bond. Examples include but are not limited to cyclopropenyl, 1-cyclobutenyl, 2-cyclobutenyl, 1-cyclopentenyl, 2-cyclopentenyl, 3-cyclopentenyl, 1-cyclohexyl, 2-cyclohexyl, 3-cyclohexyl and the like.

Alkoxy

The term “alkoxy” as used herein is an alkyl residue, as defined above, bonded directly to an oxygen atom, which is then bonded to another moiety. Examples include methoxy, ethoxy, n-propoxy, iso-propoxy, n-butoxy, t-butoxy, iso-butoxy and the like

Mono-Substituted Amino

The term “mono-substituted amino” as used herein is a moiety comprising an NH radical substituted with one organic substituent group, which include but are not limited to alkyls, substituted alkyls, cycloalkyls, aryls, or arylalkyls. Examples of mono-substituted amino groups include methylamino (—NH—CH₃); ethylamino (—NHCH₂CH₃), hydroxyethylamino (—NH—CH₂CH₂OH), and the like.

Di-Substituted Amino

The term “di-substituted amino” as used herein is a moiety comprising a nitrogen atom substituted with two organic radicals that can be the same or different, which can be selected from but are not limited to aryl, substituted aryl, alkyl, substituted alkyl or arylalkyl, wherein the terms have the same definitions found throughout. Some examples include dimethylamino, methylethylamino, diethylamino and the like.

Azide

As used herein, the term “azide”, “azido” and their variants refer to any moiety or compound comprising the monovalent group —N₃ or the monovalent ion —N₃.

Haloalkyl

The term “haloalkyl” as used herein an alkyl residue as defined above, substituted with one or more halogens, preferably fluorine, such as a trifluoromethyl, pentafluoroethyl and the like.

Haloalkoxy

The term “haloalkoxy” as used herein a haloalkyl residue as defined above that is directly attached to an oxygen to form trifluoromethoxy, pentafluoroethoxy and the like.

Acyl

The term “acyl” as used herein is a R—C(O)— residue having an R group containing 1 to 8 carbons. Examples include but are not limited to formyl, acetyl, propionyl, butanoyl, iso-butanoyl, pentanoyl, hexanoyl, heptanoyl, benzoyl and the like, and natural or un-natural amino acids.

Acyloxy

The term “acyloxy” as used herein is an acyl radical as defined above directly attached to an oxygen to form an R—C(O)O— residue. Examples include but are not limited to acetyloxy, propionyloxy, butanoyloxy, iso-butanoyloxy, benzoyloxy and the like.

Aryl

The term “aryl” as used herein is a ring radical containing 6 to 18 carbons, or preferably 6 to 12 carbons, comprising at least one aromatic residue therein. Examples of such aryl radicals include phenyl, naphthyl, and ischroman radicals. Moreover, the term “aryl” as used throughout the specification and claims is intended to include both “unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. It will be understood by those skilled in the art that the moieties substituted on the “aryl” can themselves be substituted, as described above, if appropriate.

Heteroaryl

The term “heteroaryl” as used herein is an aryl ring radical as defined above, wherein at least one of the ring carbons, or preferably 1, 2, or 3 carbons of the aryl aromatic ring has been replaced with a heteroatom, which include but are not limited to nitrogen, oxygen, and sulfur atoms. Examples of heteroaryl residues include pyridyl, bipyridyl, furanyl, and thiofuranyl residues. Substituted “heteroaryl” residues can have one or more organic or inorganic substituent groups, or preferably 1, 2, or 3 such groups, as referred to herein-above for aryl groups, bound to the carbon atoms of the heteroaromatic rings. The organic substituent groups can comprise from 1 to 12 carbon atoms, or from 1 to 6 carbon atoms, or from 1 to 4 carbon atoms.

Heterocyclyl

The term “heterocyclyl” or “heterocyclic group” as used herein is a non-aromatic mono- or multi ring radical structure having 3 to 16 members, preferably 4 to 10 members, in which at least one ring structure include 1 to 4 heteroatoms (e.g. O, N, S, P, and the like). Heterocyclyl groups include, for example, pyrrolidine, oxolane, thiolane, imidazole, oxazole, piperidine, piperizine, morpholine, lactones, lactams, such as azetidiones, and pyrrolidiones, sultams, sultones, and the like. Moreover, the term “heterocyclyl” as used throughout the specification and claims is intended to include both “unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. It will be understood by those skilled in the art that the moieties substituted on the “heterocyclyl” can themselves be substituted, as described above, if appropriate.

Halogen or Halo

The term “halo” or “halogen” refers to a fluoro, chloro, bromo or iodo group.

Moiety

A “moiety” is part of a molecule (or compound, or analog, etc.). A “functional group” is a specific group of atoms in a molecule. A moiety can be a functional group or can include one or functional groups.

Ester

The term “ester” as used herein is represented by the formula —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

Carbonate Group

The term “carbonate group” as used herein is represented by the formula —OC(O)OR, where R can be hydrogen, an alkyl, alkenyl, alkynyl, aryl, aralkyl, cycloalkyl, halogenated alkyl, or heterocycloalkyl group described above.

Keto Group

The term “keto group” as used herein is represented by the formula —C(O)R, where R is an alkyl, alkenyl, alkynyl, aryl, aralkyl, cycloalkyl, halogenated alkyl, or heterocycloalkyl group described above.

Aldehyde

The term “aldehyde” as used herein is represented by the formula —C(O)H.

Carboxylic Acid

The term “carboxylic acid” as used herein is represented by the formula —C(O)OH.

Carbonyl Group

The term “carbonyl group” as used herein is represented by the formula C═O.

Ether

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

Urethane

The term “urethane” as used herein is represented by the formula —OC(O)NRR′, where R and R′ can be, independently, hydrogen, an alkyl, alkenyl, alkynyl, aryl, aralkyl, cycloalkyl, halogenated alkyl, or heterocycloalkyl group described above.

Silyl Group

The term “silyl group” as used herein is represented by the formula —SiRR′R″, where R, R′, and R″ can be, independently, hydrogen, an alkyl, alkenyl, alkynyl, aryl, aralkyl, cycloalkyl, halogenated alkyl, alkoxy, or heterocycloalkyl group described above.

Sulfo-Oxo Group

The term “sulfo-oxo group” as used herein is represented by the formulas —S(O)₂R, —OS(O)₂R, or, —OS(O)₂OR, where R can be hydrogen, an alkyl, alkenyl, alkynyl, aryl, aralkyl, cycloalkyl, halogenated alkyl, or heterocycloalkyl group described above.

Detect

Detect or like terms refer to an ability of the apparatus and methods of the disclosure to discover or sense a molecule- or a marker-induced cellular response and to distinguish the sensed responses for distinct molecules.

Direct Action (of a Drug Candidate Molecule)

A “direct action” or like terms is a result (of a drug candidate molecule”) acting independently on a cell.

DMR Signal

A “DMR signal” or like terms refers to the signal of cells measured with an optical biosensor that is produced by the response of a cell upon stimulation.

DMR Response

A “DMR response” or like terms is a biosensor response using an optical biosensor. The DMR refers to dynamic mass redistribution or dynamic cellular matter redistribution. A P-DMR is a positive DMR response, a N-DMR is a negative DMR response, and a RP-DMR is a recovery P-DMR response.

Drug Candidate Molecule

A drug candidate molecule or like terms is a test molecule which is being tested for its ability to function as a drug or a pharmacophore. This molecule may be considered as a lead molecule.

Efficacy

Efficacy or like terms is the capacity to produce a desired size of an effect under ideal or optimal conditions. It is these conditions that distinguish efficacy from the related concept of effectiveness, which relates to change under real-life conditions. Efficacy is the relationship between receptor occupancy and the ability to initiate a response at the molecular, cellular, tissue or system level.

hERG Modulator

A hERG modulator is a molecule that can modulate the activity of hERG ion channel directly or indirectly. A hERG modulator that modulates the activity of hERG channel directly is a molecule that binds to hERG channels, thus causing the alteration in hERG activity, such as hERG current, ion flux via hERG, and/or cell signaling via hERG. A hERG modulator that modulates the activity of hERG channel indirectly is a molecule that binds to a hERG-associated signaling complex in cells, thus causing the alteration in hERG activity, such as hERG current, ion flux via hERG, and/or cell signaling via hERG channel or hERG-associated signaling complex. The alteration in hERG activity is referenced to the basal activity of hERG channel or hERG-associated signaling complex in cells in the absence of a modulator.

hERG Activator

A hERG activator is a molecule that increases the current via hERG channel at appropriate applied voltages, and/or increases the ion flux via hERG channel in the presence of appropriate KCl concentrations, and/or triggers cell signaling via hERG channel or hERG-associated signaling complex in cells. Examples are mallotoxin, flufenamic acid, and niflumic acid.

hERG Pathway Activator

A hERG pathway activator is a molecule that triggers cell signaling via hERG channel or hERG-associated signaling complex in cells. A hERG pathway activator may or may not cause any alteration in hERG current, and/or ion flux via hERG channel. Alteration can either increase or decrease. Examples are diflunisal, AG126, and tyrphostin 51.

hERG Ion Channel Activator

A hERG ion channel activator is a molecule that directly binds to and activates hERG channel, thus leading to increase in hERG current, and/or increase in hERG ion flux, and/or cell signaling via hERG channel. Examples are mallotoxin, flufenamic acid, and niflumic acid. A hERG ion channel activator may or may not trigger cell signaling.

Label-Free Biosensor hERG Activator

A label-free biosensor hERG activator or like terms is a molecule that is a hERG activator and is capable of triggering a detectable biosensor signal in a hERG expressing cell using a label-free biosensor cellular assay. The biosensor hERG activator can be a hERG activator, a hERG pathway activator, or a hERG ion channel activator. Examples are mallotoxin, RPR260243, NS1643, NS3623, PD-118057, PD-307243, A-935142, flufenamic acid, niflumic acid, or diflunisal.

hERG Inhibitor

A hERG inhibitor is a molecule that binds to hERG channel, or hERG-associated signaling complex, thus inhibiting hERG current and/or hERG ion flux.

hERG Pathway Inhibitor

A hERG inhibitor is a molecule that binds to hERG-associated signaling complex, thus inhibiting hERG current, and/or hERG ion flux. Example includes tyrphostin 51.

hERG Ion Channel Inhibitor

A hERG ion channel inhibitor is a molecule that binds to hERG channel directly and thus inhibits hERG current, and/or hERG ion flux. Example includes dofetilide.

Higher and Inhibit and Like Words

The terms higher, increases, elevates, or elevation or like terms or variants of these terms, refer to increases above basal levels, e.g., as compared a control. The terms low, lower, reduces, decreases or reduction or like terms or variation of these terms, refer to decreases below basal levels, e.g., as compared to a control. For example, basal levels are normal in vivo levels prior to, or in the absence of, or addition of a molecule such as an agonist or antagonist to a cell Inhibit or forms of inhibit or like terms refers to reducing or suppressing.

In the Presence of the Molecule

“in the presence of the molecule” or like terms refers to the contact or exposure of the cultured cell with the molecule. The contact or exposure can be taken place before, or at the time, the stimulus is brought to contact with the cell.

Index

An index or like terms is a collection of data. For example, an index can be a list, table, file, or catalog that contains one or more modulation profiles. It is understood that an index can be produced from any combination of data. For example, a DMR profile can have a P-DMR, a N-DMR, and a RP-DMR. An index can be produced using the completed date of the profile, the P-DMR data, the N-DMR data, the RP-DMR data, or any point within these, or in combination of these or other data. The index is the collection of any such information. Typically, when comparing indexes, the indexes are of like data, i.e. P-DMR to P-DMR Data.

Biosensor Index

A “biosensor index” or like terms is an index made up of a collection of biosensor data. A biosensor index can be a collection of biosensor profiles, such as primary profiles, or secondary profiles. The index can be comprised of any type of data. For example, an index of profiles could be comprised of just an N-DMR data point, it could be a P-DMR data point, or both or it could be an impedence data point. It could be all of the data points associated with the profile curve.

DMR Index

A “DMR index” or like terms is a biosensor index made up of a collection of DMR data.

Known Molecule

A known molecule or like terms is a molecule with known pharmacological/biological/physiological/pathophysiological activity whose precise mode of action(s) may be known or unknown.

Known Modulator

A known modulator or like terms is a modulator where at least one of the targets is known with a known affinity. For example, a known modulator could be a PI3K inhibitor, a PKA inhibitor, a GPCR antagonist, a GPCR agonist, a RTK inhibitor, an epidermal growth factor receptor neutralizing antibody, or a phosphodiesterase inhibition, a PKC inhibitor or activator, etc.

Known Modulator Bio Sensor Index

A “known modulator biosensor index” or like terms is a modulator biosensor index produced by data collected for a known modulator. For example, a known modulator biosensor index can be made up of a profile of the known modulator acting on the panel of cells, and the modulation profile of the known modulator against the panels of markers, each panel of markers for a cell in the panel of cells.

Known Modulator DMR Index

A “known modulator DMR index” or like terms is a modulator DMR index produced by data collected for a known modulator. For example, a known modulator DMR index can be made up of a profile of the known modulator acting on the panel of cells, and the modulation profile of the known modulator against the panels of markers, each panel of markers for a cell in the panel of cells.

Ligand

A ligand or like terms is a substance or a composition or a molecule that is able to bind to and form a complex with a biomolecule to serve a biological purpose. Actual irreversible covalent binding between a ligand and its target molecule is rare in biological systems. Ligand binding to receptors alters the chemical conformation, i.e., the three dimensional shape of the receptor protein. The conformational state of a receptor protein determines the functional state of the receptor. The tendency or strength of binding is called affinity. Ligands include substrates, blockers, inhibitors, activators, and neurotransmitters. Radioligands are radioisotope labeled ligands, while fluorescent ligands are fluorescently tagged ligands; both can be considered as ligands are often used as tracers for receptor biology and biochemistry studies. Ligand and modulator are used interchangeably.

Library

A library or like terms is a collection. The library can be a collection of anything disclosed herein. For example, it can be a collection, of indexes, an index library; it can be a collection of profiles, a profile library; or it can be a collection of DMR indexes, a DMR index library; Also, it can be a collection of molecule, a molecule library; it can be a collection of cells, a cell library; it can be a collection of markers, a marker library; A library can be for example, random or non-random, determined or undetermined. For example, disclosed are libraries of DMR indexes or biosensor indexes of known modulators.

Marker

A marker or like terms is a ligand which produces a signal in a biosensor cellular assay. The signal is, must also be, characteristic of at least one specific cell signaling pathway(s) and/or at least one specific cellular process(es) mediated through at least one specific target(s). The signal can be positive, or negative, or any combinations (e.g., oscillation). A hERG channel activator, such as mallotoxin, can be a marker for HEK-hERG cells, or HT29 cells, wherein hERG channels are stably expressed, or endogenously expressed in respective cells.

Marker Panel

A “marker panel” or like terms is a panel which comprises at least two markers.

The markers can be for different pathways, the same pathway, different targets, or even the same targets. For example, mallotoxin can be used as a single marker for both HEK-hERG and HT29 cells. Thus for hERG channel modulator identification and classification, mallotoxin acts as an effective marker panel.

Marker Biosensor Index

A “marker biosensor index” or like terms is a biosensor index produced by data collected for a marker. For example, a marker biosensor index can be made up of a profile of the marker acting on the panel of cells, and the modulation profile of the marker against the panels of markers, each panel of markers for a cell in the panel of cells. For hERG channel modulator identification and classification, the marker biosensor index includes the primary profiles of mallotoxin across three different cells (e.g., HEK293, HEK-hERG, and HT29 cells), and the modulation index of mallotoxin against the mallotoxin DMR signals in both HEK-hERG and HT29 cells, as exampled in FIG. 1.

Marker DMR Index

A “marker biosensor index” or like terms is a biosensor DMR index produced by data collected for a marker. For example, a marker DMR index can be made up of a profile of the marker acting on the panel of cells, and the modulation profile of the marker against the panels of markers, each panel of markers for a cell in the panel of cells.

Material

Material is the tangible part of something (chemical, biochemical, biological, or mixed) that goes into the makeup of a physical object.

Mimic

As used herein, “mimic” or like terms refers to performing one or more of the functions of a reference object. For example, a molecule mimic performs one or more of the functions of a molecule.

Modulate

To modulate, or forms thereof, means either increasing, decreasing, or maintaining a cellular activity mediated through a cellular target. It is understood that wherever one of these words is used it is also disclosed that it could be 1%, 5%, 10%, 20%, 50%, 100%, 500%, or 1000% increased from a control, or it could be 1%, 5%, 10%, 20%, 50%, or 100% decreased from a control.

Modulator

A modulator or like terms is a ligand that controls the activity of a cellular target. It is a signal modulating molecule binding to a cellular target, such as a target protein.

Modulation Comparison

A “modulation comparison” or like terms is a result of normalizing a primary profile and a secondary profile.

Modulator Biosensor Index

A “modulator biosensor index” or like terms is a biosensor index produced by data collected for a modulator. For example, a modulator biosensor index can be made up of a profile of the modulator acting on the panel of cells, and the modulation profile of the modulator against the panels of markers, each panel of markers for a cell in the panel of cells. As exampled in FIGS. 2 to 5, a hERG modulator biosensor index includes the primary DMR signals in three types of cells (e.g., HT29, HEK-hERG, and HEK293), and the modulation DMR index of the modulator against the mallotoxin DMR signals in both HT29 and HEK-hERG cells.

Modulator DMR Index

A “modulator DMR index” or like terms is a DMR index produced by data collected for a modulator. For example, a modulator DMR index can be made up of a profile of the modulator acting on the panel of cells, and the modulation profile of the modulator against the panels of markers, each panel of markers for a cell in the panel of cells. As exampled in FIGS. 1d to 5d, a hERG modulator DMR index is the percentage in modulation of the mallotoxin DMR signals in both HT29 and HEK-hERG cells by the modulator.

Modulate the Biosensor Signal of a Marker

Modulate the biosensor signal or like terms is to cause changes of the biosensor signal or profile of a cell in response to stimulation with a marker.

Modulate the DMR Signal

“Modulate the DMR signal or like terms is to cause changes of the DMR signal or profile of a cell in response to stimulation with a marker.

Molecule

As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

Molecule Mixture

A molecule mixture or like terms is a mixture containing at least two molecules. The two molecules can be, but not limited to, structurally different (i.e., enantiomers), or compositionally different (e.g., protein isoforms, glycoform, or an antibody with different poly(ethylene glycol) (PEG) modifications), or structurally and compositionally different (e.g., unpurified natural extracts, or unpurified synthetic compounds).

Molecule Biosensor Index

A “molecule biosensor index” or like terms is a biosensor index produced by data collected for a molecule. For example, a molecule biosensor index can be made up of a profile of the molecule acting on the panel of cells, and the modulation profile of the molecule against the panels of markers, each panel of markers for a cell in the panel of cells.

Molecule DMR Index

A “molecule DMR index” or like terms is a DMR index produced by data collected for a molecule. For example, a molecule biosensor index can be made up of a profile of the molecule acting on the panel of cells, and the modulation profile of the molecule against the panels of markers, each panel of markers for a cell in the panel of cells.

Molecule Index

A “molecule index” or like terms is an index related to the molecule.

Molecule-Treated Cell

A molecule-treated cell or like terms is a cell that has been exposed to a molecule.

Molecule Modulation Index

A “molecule modulation index” or like terms is an index to display the ability of the molecule to modulate the biosensor output signals of the panels of markers acting on the panel of cells. The modulation index is generated by normalizing a specific biosensor output signal parameter of a response of a cell upon stimulation with a marker in the presence of a molecule against that in the absence of any molecule.

Molecule Pharmacology

Molecule pharmacology or the like terms refers to the systems cell biology or systems cell pharmacology or mode(s) of action of a molecule acting on a cell. The molecule pharmacology is often characterized by, but not limited, toxicity, ability to influence specific cellular process(es) (e.g., proliferation, differentiation, reactive oxygen species signaling), or ability to modulate a specific cellular target (e.g, hERG channel, hERG-associated signaling complex, PI3K, PKA, PKC, PKG, JAK2, MAPK, MEK2, or actin).

Normalizing

Normalizing or like terms means, adjusting data, or a profile, or a response, for example, to remove at least one common variable. For example, if two responses are generated, one for a marker acting a cell and one for a marker and molecule acting on the cell, normalizing would refer to the action of comparing the marker-induced response in the absence of the molecule and the response in the presence of the molecule, and removing the response due to the marker only, such that the normalized response would represent the response due to the modulation of the molecule against the marker. A modulation comparison is produced by normalizing a primary profile of the marker and a secondary profile of the marker in the presence of a molecule (modulation profile).

Optional

“Optional” or “optionally” or like terms means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

Or

The word “or” or like terms as used herein means any one member of a particular list and also includes any combination of members of that list.

profile

A profile or like terms refers to the data which is collected for a composition, such as a cell. A profile can be collected from a label free biosensor as described herein.

Primary Profile

A “primary profile” or like terms refers to a biosensor response or biosensor output signal or profile which is produced when a molecule contacts a cell. Typically, the primary profile is obtained after normalization of initial cellular response to the net-zero biosensor signal (i.e., baseline)

Secondary Profile

A “secondary profile” or like terms is a biosensor response or biosensor output signal of cells in response to a marker in the presence of a molecule. A secondary profile can be used as an indicator of the ability of the molecule to modulate the marker-induced cellular response or biosensor response.

Modulation Profile

A “modulation profile” or like terms is the comparison between a secondary profile of the marker in the presence of a molecule and the primary profile of the marker in the absence of any molecule. The comparison can be by, for example, subtracting the primary profile from secondary profile or subtracting the secondary profile from the primary profile or normalizing the secondary profile against the primary profile.

Panel

A panel or like terms is a predetermined set of specimens (e.g., markers, or cells, or pathways). A panel can be produced from picking specimens from a library.

Positive Control

A “positive control” or like terms is a control that shows that the conditions for data collection can lead to data collection.

Potentiate

Potentiate, potentiated or like terms refers to an increase of a specific parameter of a biosensor response of a marker in a cell caused by a molecule. By comparing the primary profile of a marker with the secondary profile of the same marker in the same cell in the presence of a molecule, one can calculate the modulation of the marker-induced biosensor response of the cells by the molecule. A positive modulation means the molecule to cause increase in the biosensor signal induced by the marker.

Potency

Potency or like terms is a measure of molecule activity expressed in terms of the amount required to produce an effect of given intensity. For example, a highly potent drug evokes a larger response at low concentrations. The potency is proportional to affinity and efficacy. Affinity is the ability of the drug molecule to bind to a receptor.

Publications

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Receptor

A receptor or like terms is a protein molecule embedded in either the plasma membrane or cytoplasm of a cell, to which a mobile signaling (or “signal”) molecule may attach. A molecule which binds to a receptor is called a “ligand,” and may be a peptide (such as a neurotransmitter), a hormone, a pharmaceutical drug, or a toxin, and when such binding occurs, the receptor goes into a conformational change which ordinarily initiates a cellular response. However, some ligands merely block receptors without inducing any response (e.g. antagonists). Ligand-induced changes in receptors result in physiological changes which constitute the biological activity of the ligands.

“Robust Biosensor Signal”

A “robust biosensor signal” is a biosensor signal whose amplitude(s) is significantly (such as 3×, 10×, 20×, 100×, or 1000×) above either the noise level, or the negative control response. The negative control response is often the biosensor response of cells after addition of the assay buffer solution (i.e., the vehicle). The noise level is the biosensor signal of cells without further addition of any solution. It is worthy of noting that the cells are always covered with a solution before addition of any solution.

“Robust DMR Signal”

A “robust DMR signal” or like terms is a DMR form of a “robust biosensor signal.”

Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Response

A response or like terms is any reaction to any stimulation.

Sample

By sample or like terms is meant an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

Signaling Pathway(s)

A “defined pathway” or like terms is a path of a cell from receiving a signal (e.g., an exogenous ligand) to a cellular response (e.g., increased expression of a cellular target). In some cases, receptor activation caused by ligand binding to a receptor is directly coupled to the cell's response to the ligand. For example, the neurotransmitter GABA can activate a cell surface receptor that is part of an ion channel. GABA binding to a GABA A receptor on a neuron opens a chloride-selective ion channel that is part of the receptor. GABA A receptor activation allows negatively charged chloride ions to move into the neuron which inhibits the ability of the neuron to produce action potentials. However, for many cell surface receptors, ligand-receptor interactions are not directly linked to the cell's response. The activated receptor must first interact with other proteins inside the cell before the ultimate physiological effect of the ligand on the cell's behavior is produced. Often, the behavior of a chain of several interacting cell proteins is altered following receptor activation. The entire set of cell changes induced by receptor activation is called a signal transduction mechanism or pathway. The signaling pathway can be either relatively simple or quite complicated.

Similarity of Indexes

“Similarity of indexes” or like terms is a term to express the similarity between two indexes, or among at least three indices, one for a molecule, based on the patterns of indices, and/or a matrix of scores. The matrix of scores are strongly related to their counterparts, such as the signatures of the primary profiles of different molecules in corresponding cells, and the nature and percentages of the modulation profiles of different molecules against each marker. For example, higher scores are given to more-similar characters, and lower or negative scores for dissimilar characters. Because there are only three types of modulation, positive, negative and neutral, found in the molecule modulation index, the similarity matrices are relatively simple. For example, a simple matrix will assign identical modulation (e.g., a positive modulation) a score of +1 and non-identical modulation a score of −1.

Alternatively, different scores can be given for a type of modulation but with different scales. For example, a positive modulation of 10%, 20%, 30%, 40%, 50%, 60%, 100%, 200%, etc, can be given a score of +1, +2, +3, +4, +5, +6, +10, +20, correspondingly. Conversely, for negative modulation, similar but in opposite score can be given. Following this approach, the modulation index of flufenamic acid against mallotoxin in the two cells, as shown in FIG. 2d, illustrates that flufenamic acid modulates differently the biosensor response induced by mallotoxin in the two cells: HT29 (−90%), and HEK-hERG (˜+12%). Thus, the score of flufenamic acid modulation index in coordination can be assigned as (−9, 1). Similarly, for diflunisal its score in coordination is (−9, 1). By comparing the scores between flufenamic acid and diflunisal, one can conclude that both molecules exhibits similar mode(s) of action acting on hERG channels.

Stable

When used with respect to pharmaceutical compositions, the term “stable” or like terms is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

Substance

A substance or like terms is any physical object. A material is a substance. Molecules, ligands, markers, cells, proteins, and DNA can be considered substances. A machine or an article would be considered to be made of substances, rather than considered a substance themselves.

Subject

As used throughout, by a subject or like terms is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) and mammals, non-human mammals, primates, non-human primates, rodents, birds, reptiles, amphibians, fish, and any other animal. In one aspect, the subject is a mammal such as a primate or a human. The subject can be a non-human.

Test Molecule

A test molecule or like terms is a molecule which is used in a method to gain some information about the test molecule. A test molecule can be an unknown or a known molecule.

Treating

Treating or treatment or like terms can be used in at least two ways. First, treating or treatment or like terms can refer to administration or action taken towards a subject. Second, treating or treatment or like terms can refer to mixing any two things together, such as any two or more substances together, such as a molecule and a cell. This mixing will bring the at least two substances together such that a contact between them can take place.

When treating or treatment or like terms is used in the context of a subject with a disease, it does not imply a cure or even a reduction of a symptom for example. When the term therapeutic or like terms is used in conjunction with treating or treatment or like terms, it means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

Trigger

A trigger or like terms refers to the act of setting off or initiating an event, such as a response.

Values

Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

Thus, the disclosed methods, compositions, articles, and machines, can be combined in a manner to comprise, consist of, or consist essentially of, the various components, steps, molecules, and composition, and the like, discussed herein. They can be used, for example, in methods for characterizing a molecule including a ligand as defined herein; a method of producing an index as defined herein; or a method of drug discovery as defined herein.

Unknown Molecule

An unknown molecule or like terms is a molecule with unknown biological/pharmacological/physiological/pathophysiological activity.

### Voltage-Dependent Ion Channels

Voltage-dependant ion channels are proteins that span cell surface membranes in excitable tissue such as heart and nerves. Ions passing through channels form the basis of the cardiac action potential. Influx of Na⁺ and Ca²⁺ ions, respectively, control the depolarizing upstroke and plateau phases of the action potential. K⁺ ion efflux repolarizes the cell membrane, terminates the action potential, and allows relaxation of the muscle. A rapid component of the repolarizing current flows through the K⁺ channel encoded by the human ether-a-go-go-related gene (hERG). Impaired repolarization can prolong the duration of the action potential, delay relaxation and promote disturbances of the heartbeat. Action potential prolongation is detected clinically as a lengthening of the QT interval measured on the electrocardiogram (ECG). Drug-induced QT prolongation is a serious complication of drugs due to impaired repolarization, which is associated with an increased risk of lethal ventricular arrhythmias. Drug-induced QT prolongation is almost always associated with block of the hERG K⁺ channel. A plethora of drugs, such as methanesulfonanilides, dofetilide, MK-499, and E-4031 are known to block K⁺ ion channels such as hERG on the heart causing a life threatening ventricular arrhythmia and heart attack in susceptible individuals. Unfortunately, incidence of drug-induced ventricular arrhythmia is often too low to be detected in clinical trials.

A sudden death due to the blocking of hERG channels by noncardiovascular drugs such as terfenadine (antihistamine), astemizole (antihistamine), and cisapride (gastrokinetic) led to their withdrawal from the market. Recently, drugs like Vioxx were also pulled out of the market for concerns relating to dangerous cardiac side effects. Consequently, cardiac safety relating to K⁺ channels has become a major concern of regulatory agencies. In order to prevent costly attrition, it has therefore become a high priority in drug discovery to screen out inhibitory activity on hERG channels in lead compounds as early as possible.

Current methods for testing potential drug molecules for hERG blocking activity have several limitations. Technologies based on cell-based patch clamp electrophysiology or animal tests are technically difficult and do not meet the demand for throughput and precision for preclinical cardiac safety tests. Other assays use radio-labeled, fluorescent, dye-conjugated, or biotinylated markers for detection and quantification of binding. However, many of these markers have reduced activity after labeling. In addition, the use of radio-labeled analogs poses practical limitations such as requirements for complex infrastructure and licenses for operating radioactive compounds. The promiscuous nature of this channel, referred to herein as the hERG K⁺ channel, or hERG, or hERG ion channel, or hERG channel, leads to it binding a diverse set of chemical structures (Cavalli, A., et al., J. Med. Chem. 2002, 45(18), 3844-53), coupled with the potential fatal outcome that may emerge from that interaction. These realities have resulted in the recommendation from the International Congress of Harmonization and the U.S. Food and Drug Administration that all new drug candidates undergo testing in a functional patch-clamp assay using the human hERG protein, either in native form or expressed in recombinant form (Bode, G., et al., Fundam. Clin. Pharmacol. 2002, 16(2), 105-18). Although automated, high-throughput patch-clamp methods have been recently developed, such systems require specialized operators, live cells, and a substantial capital investment (Bridgland-Taylor, M., et al., J. Pharmacol. Toxicol. Methods 2006, 54(2), 189-99; Dubin, A., et al., J. Biomol. Screen. 2005, 10(2), 168-81). Accordingly, there is a need to develop new compositions and methods for characterizing and quantifying the binding of molecules, such as drug candidates, to hERG channels.

The KCNH2 or human-Ether-a-go-go Related Gene (hERG) encodes Kv11.1 α-subunits that combine to form Kv11.1 potassium channels. The hERG gene is translated as a core-glycosylated immature 135 kDa protein (Kv11.1) in the endoplasmic reticulum and is converted to a complexly-glycosylated mature 155 kDa protein in the Golgi apparatus, Warmke, J. W., et al. (Proc. Natl. Acad. Sci. USA 1994, 91(8), 3438-3442; incorporated by reference) discloses the sequence and structure of the hERG gene and its wild type translation product, Kv11.1. The sequence of hERG protein is disclosed in SEQ ID NO: NP_{—}000229. The sequence of hERG gene is disclosed in SEQ ID NO: NM_{—}00238.

### Assays

Functional hERG Ion Channel Assays with High Throughput

Disclosed are methods for assaying both endogenous and stably over-expressed hERG ion channels and screen hERG modulators with high throughput and high resolution. Label-free biosensor cellular assays have been shown to be able to study the biology of receptors in live cells, and provide a functional readout (e.g., dynamic mass redistribution signal). The resultant DMR signal is an integrated cellular response, and follows the entire evolution of ion channel activity. The real time kinetics enables classification of modes of action of modulators acting on ion channels and their regulatory proteins.

Since label-free RWG biosensors measure an integrated and kinetic response of cells upon stimulation with ion channel modulators, the disclosed biosensor assays provide a new way to classify hERG modulators, particularly for those being capable of activating signaling via hERG channels or hERG-associated signaling complexes, but with or without impacting the hERG current and/or the ion flux via hERG channels.

Combining multiplexed label-free readouts in different cells in the absence or presence of a hERG activator (e.g., mallotoxin) offers effective ways to classify hERG modulators into different categories.

The present methods and compositions are related to label-free biosensor cellular assays. A label-free biosensor for cellular assays is a device consisting of three components—a biological component (i.e., live cells), a detector element, and a transducer associated with both components. Depending on the types of transducers used, label-free biosensors for whole cell sensing can be largely classified into three categories: acoustic, electrical and optical biosensors.

### Biosensors

### Acoustic Biosensors

Acoustic biosensors such as quartz crystal resonators utilize acoustic waves to characterize cellular responses. The acoustic waves are generally generated and received using piezoelectric. An acoustic biosensor is often designed to operate in a resonant type sensor configuration. In a typical setup, thin quartz discs are sandwiched between two gold electrodes. Application of an AC signal across electrodes leads to the excitation and oscillation of the crystal, which acts as a sensitive oscillator circuit. The output sensor signals are the resonance frequency and motional resistance. The resonance frequency is largely a linear function of total mass of adsorbed materials when the biosensor surface is rigid. Under liquid environments the acoustic sensor response is sensitive not only to the mass of bound molecules, but also to changes in viscoelastic properties and charge of the molecular complexes formed or live cells. By measuring the resonance frequency and the motion resistance of cells associated with the crystals, cellular processes including cell adhesion and cytotoxicity can be studied in real time.

### Electrical Biosensors

Electrical biosensors employ impedance to characterize cellular responses including cell adhesion. In a typical setup, live cells are brought in contact with a biosensor surface wherein an integrated electrode array is embedded. A small AC pulse at a constant voltage and high frequency is used to generate an electric field between the electrodes, which are impeded by the presence of cells. The electric pulses are generated on site using the integrated electric circuit; and the electrical current through the circuit is followed with time. The resultant impedance is a measure of changes in the electrical conductivity of the cell layer. The cellular plasma membrane acts as an insulating agent forcing the current to flow between or beneath the cells, leading to quite robust changes in impedance. Impedance-based measurements have been applied to study a wide range of cellular events, including cell adhesion and spreading, cell micromotion, cell morphological changes, and cell death, and cell signaling.

### Optical Biosensors

Optical biosensors primarily employ a surface-bound electromagnetic wave to characterize cellular responses. The surface-bound waves can be achieved either on gold substrates using either light excited surface plasmons (surface plasmon resonance, SPR) or on dielectric substrate using diffraction grating coupled waveguide mode resonances (resonance waveguide grating, RWG). For SPR, the readout is the resonance angle at which a minimal in intensity of reflected light occurs. Similarly, for RWG biosensor, the readout is the resonance angle or wavelength at which a maximum incoupling efficiency is achieved. The resonance angle or wavelength is a function of the local refractive index at or near the sensor surface. Unlike SPR, which is limited to a few of flow channels for assaying, RWG biosensors are amenable for high throughput screening (HTS) and cellular assays, due to recent advancements in instrumentation and assays. In a typical RWG, the cells are directly placed into a well of a microtiter plate in which a biosensor consisting of a material with high refractive index is embedded. Local changes in the refractive index lead to a dynamic mass redistribution (DMR) signal of live cells upon stimulation. These biosensors have been used to study diverse cellular processes including receptor biology, ligand pharmacology, and cell adhesion.

The present invention preferably uses resonant waveguide grating biosensors, such as Corning Epic® systems. Epic® system includes the commercially available wavelength integration system, or angular interrogation system or swept wavelength imaging system (Corning Inc., Corning, N.Y.). The commercial system consists of a temperature-control unit, an optical detection unit, with an on-board liquid handling unit with robotics, or an external liquid accessory system with robotics. The detection unit is centered on integrated fiber optics, and enables kinetic measures of cellular responses with a time interval of ˜7 or 15 sec. The compound solutions were introduced by using either the on-board liquid handling unit, or the external liquid accessory system; both of which use conventional liquid handling system.

### Biosensors and Biosensor Assays

Label-free cell-based assays generally employ a biosensor to monitor molecule-induced responses in living cells. The molecule can be naturally occurring or synthetic, and can be a purified or unpurified mixture. A biosensor typically utilizes a transducer such as an optical, electrical, calorimetric, acoustic, magnetic, or like transducer, to convert a molecular recognition event or a molecule-induced change in cells contacted with the biosensor into a quantifiable signal. These label-free biosensors can be used for molecular interaction analysis, which involves characterizing how molecular complexes form and disassociate over time, or for cellular response, which involves characterizing how cells respond to stimulation. The biosensors that are applicable to the present methods can include, for example, optical biosensor systems such as surface plasmon resonance (SPR) and resonant waveguide grating (RWG) biosensors, resonant mirrors, ellipsometers, and electric biosensor systems such as bioimpedance systems.

SPR Biosensors and Systems

SPR relies on a prism to direct a wedge of polarized light, covering a range of incident angles, into a planar glass substrate bearing an electrically conducting metallic film (e.g., gold) to excite surface plasmons. The resultant evanescent wave interacts with, and is absorbed by, free electron clouds in the gold layer, generating electron charge density waves (i.e., surface plasmons) and causing a reduction in the intensity of the reflected light. The resonance angle at which this intensity minimum occurs is a function of the refractive index of the solution close to the gold layer on the opposing face of the sensor surface

RWG Biosensors and Systems

An RWG biosensor can include, for example, a substrate (e.g., glass), a waveguide thin film with an embedded grating or periodic structure, and a cell layer. The RWG biosensor utilizes the resonant coupling of light into a waveguide by means of a diffraction grating, leading to total internal reflection at the solution-surface interface, which in turn creates an electromagnetic field at the interface. This electromagnetic field is evanescent in nature, meaning that it decays exponentially from the sensor surface; the distance at which it decays to 1/e of its initial value is known as the penetration depth and is a function of the design of a particular RWG biosensor, but is typically on the order of about 200 nm. This type of biosensor exploits such evanescent wave to characterize ligand-induced alterations of a cell layer at or near the sensor surface.

RWG instruments can be subdivided into systems based on angle-shift or wavelength-shift measurements. In a wavelength-shift measurement, polarized light covering a range of incident wavelengths with a constant angle is used to illuminate the waveguide; light at specific wavelengths is coupled into and propagates along the waveguide. Alternatively, in angle-shift instruments, the sensor is illuminated with monochromatic light and the angle at which the light is resonantly coupled is measured.

The resonance conditions are influenced by the cell layer (e.g., cell confluency, adhesion and status), which is in direct contact with the surface of the biosensor. When a ligand or an analyte interacts with a cellular target (e.g., a GPCR, an ion channel, a kinase) in living cells, any change in local refractive index within the cell layer can be detected as a shift in resonant angle (or wavelength).

The Corning® Epic® system uses RWG biosensors for label-free biochemical or cell-based assays (Corning Inc., Corning, N.Y.). The Epic® System consists of an RWG plate reader and SBS (Society for Biomolecular Screening) standard microtiter plates. The detector system in the plate reader exploits integrated fiber optics to measure the shift in wavelength of the incident light, as a result of ligand-induced changes in the cells. A series of illumination-detection heads are arranged in a linear fashion, so that reflection spectra are collected simultaneously from each well within a column of a 384-well microplate. The whole plate is scanned so that each sensor can be addressed multiple times, and each column is addressed in sequence. The wavelengths of the incident light are collected and used for analysis. A temperature-controlling unit can be included in the instrument to minimize spurious shifts in the incident wavelength due to the temperature fluctuations. The measured response represents an averaged response of a population of cells. Varying features of the systems can be automated, such as sample loading, and can be multiplexed, such as with a 96 or 386 well microtiter plate. Liquid handling is carried out by either on-board liquid handler, or an external liquid handling accessory. Specifically, molecule solutions are directly added or pipetted into the wells of a cell assay plate having cells cultured in the bottom of each well. The cell assay plate contains certain volume of assay buffer solution covering the cells. A simple mixing step by pipetting up and down certain times can also be incorporated into the molecule addition step.

Electrical Biosensors and Systems

Electrical biosensors consist of a substrate (e.g., plastic), an electrode, and a cell layer. In this electrical detection method, cells are cultured on small gold electrodes arrayed onto a substrate, and the system's electrical impedance is followed with time. The impedance is a measure of changes in the electrical conductivity of the cell layer. Typically, a small constant voltage at a fixed frequency or varied frequencies is applied to the electrode or electrode array, and the electrical current through the circuit is monitored over time. The ligand-induced change in electrical current provides a measure of cell response. Impedance measurement for whole cell sensing was first realized in 1984. Since then, impedance-based measurements have been applied to study a wide range of cellular events, including cell adhesion and spreading, cell micromotion, cell morphological changes, and cell death. Classical impedance systems suffer from high assay variability due to use of a small detection electrode and a large reference electrode. To overcome this variability, the latest generation of systems, such as the CellKey system (MDS Sciex, South San Francisco, Calif.) and RT-CES (ACEA Biosciences Inc., San Diego, Calif.), utilize an integrated circuit having a microelectrode array.

High Spatial Resolution Biosensor Imaging Systems

Optical biosensor imaging systems, including SPR imaging systems, ellipsometry imaging systems, and RWG imaging systems, offer high spatial resolution, and can be used in embodiments of the disclosure. For example, SPR Imager®II (GWC Technologies Inc) uses prism-coupled SPR, and takes SPR measurements at a fixed angle of incidence, and collects the reflected light with a CCD camera. Changes on the surface are recorded as reflectivity changes. Thus, SPR imaging collects measurements for all elements of an array simultaneously.

A swept wavelength optical interrogation system based on RWG biosensor for imaging-based application can be employed. In this system, a fast tunable laser source is used to illuminate a sensor or an array of RWG biosensors in a microplate format. The sensor spectrum can be constructed by detecting the optical power reflected from the sensor as a function of time as the laser wavelength scans, and analysis of the measured data with computerized resonant wavelength interrogation modeling results in the construction of spatially resolved images of biosensors having immobilized receptors or a cell layer. The use of an image sensor naturally leads to an imaging based interrogation scheme. 2 dimensional label-free images can be obtained without moving parts.

Alternatively, angular interrogation system with transverse magnetic or p-polarized TM₀ mode can also be used. This system consists of a launch system for generating an array of light beams such that each illuminates a RWG sensor with a dimension of approximately 200 μm×3000 μm or 200 μm×2000 μm, and a CCD camera-based receive system for recording changes in the angles of the light beams reflected from these sensors. The arrayed light beams are obtained by means of a beam splitter in combination with diffractive optical lenses. This system allows up to 49 sensors (in a 7×7 well sensor array) to be simultaneously sampled at every 3 seconds, or up to the whole 384 well microplate to be simultaneously sampled at every 10 seconds.

Alternatively, a scanning wavelength interrogation system can also be used. In this system, a polarized light covering a range of incident wavelengths with a constant angle is used to illuminate and scan across a waveguide grating biosensor, and the reflected light at each location can be recorded simultaneously. Through scanning, a high resolution image across a biosensor can also be achieved

Dynamic Mass Redistribution (DMR) Signals in Living Cells

The cellular response to stimulation through a cellular target can be encoded by the spatial and temporal dynamics of downstream signaling networks. For this reason, monitoring the integration of cell signaling in real time can provide physiologically relevant information that is useful in understanding cell biology and physiology.

Optical biosensors including resonant waveguide grating (RWG) biosensors, can detect an integrated cellular response related to dynamic redistribution of cellular matters, thus providing a non-invasive means for studying cell signaling. All optical biosensors are common in that they can measure changes in local refractive index at or very near the sensor surface. In principle, almost all optical biosensors are applicable for cell sensing, as they can employ an evanescent wave to characterize ligand-induced change in cells. The evanescent-wave is an electromagnetic field, created by the total internal reflection of light at a solution-surface interface, which typically extends a short distance (hundreds of nanometers) into the solution at a characteristic depth known as the penetration depth or sensing volume.

Recently, theoretical and mathematical models have been developed that describe the parameters and nature of optical signals measured in living cells in response to stimulation with ligands. These models, based on a 3-layer waveguide system in combination with known cellular biophysics, link the ligand-induced optical signals to specific cellular processes mediated through a receptor.

Because biosensors measure the average response of the cells located at the area illuminated by the incident light, a highly confluent layer of cells can be used to achieve optimal assay results. Due to the large dimension of the cells as compared to the short penetration depth of a biosensor, the sensor configuration is considered as a non-conventional three-layer system: a substrate, a waveguide film with a grating structure, and a cell layer. Thus, a ligand-induced change in effective refractive index (i.e., the detected signal) can be, to first order, directly proportional to the change in refractive index of the bottom portion of the cell layer:

ΔN=S(C)Δn_{c}

where S(C) is the sensitivity to the cell layer, and Δn_{c} the ligand-induced change in local refractive index of the cell layer sensed by the biosensor. Because the refractive index of a given volume within a cell is largely determined by the concentrations of bio-molecules such as proteins, Δn_{c} can be assumed to be directly proportional to ligand-induced change in local concentrations of cellular targets or molecular assemblies within the sensing volume. Considering the exponentially decaying nature of the evanescent wave extending away from the sensor surface, the ligand-induced optical signal is governed by:

$$Δ N = S ( C ) ad {\sum }_{i} Δ {C}_{i} [ {e}^{\frac{- {z}_{i}}{Δ {Z}_{C}}} - {e}^{\frac{- {z}_{i + 1}}{Δ {Z}_{C}}} ]$$

where ΔZ_{c} is the penetration depth into the cell layer, a the specific refraction increment (about 0.18/mL/g for proteins), zᵢ the distance where the mass redistribution occurs, and d an imaginary thickness of a slice within the cell layer. Here the cell layer is divided into an equal-spaced slice in the vertical direction. The equation above indicates that the ligand-induced optical signal is a sum of mass redistribution occurring at distinct distances away from the sensor surface, each with an unequal contribution to the overall response. Furthermore, the detected signal, in terms of wavelength or angular shifts, is primarily sensitive to mass redistribution occurring perpendicular to the sensor surface. Because of its dynamic nature, it also is referred to as dynamic mass redistribution (DMR) signal.

### Cells and Biosensors

Cells rely on multiple cellular pathways or machineries to process, encode and integrate the information they receive. Unlike the affinity analysis with optical biosensors that specifically measures the binding of analytes to a protein target, living cells are much more complex and dynamic.

To study cell signaling, cells can be brought into contact with the surface of a biosensor, which can be achieved through cell culture. These cultured cells can be attached onto the biosensor surface through three types of contacts: focal contacts, close contacts and extracellular matrix contacts, each with its own characteristic separation distance from the surface. As a result, the basal cell membranes are generally located away from the surface by ˜10-100 nm. For suspension cells, the cells can be brought in contact with the biosensor surface through either covalent coupling of cell surface receptors, or specific binding of cell surface receptors, or simply settlement by gravity force. For this reason, biosensors are able to sense the bottom portion of cells.

Cells, in many cases, exhibit surface-dependent adhesion and proliferation. In order to achieve robust cell assays, the biosensor surface can require a coating to enhance cell adhesion and proliferation. However, the surface properties can have a direct impact on cell biology. For example, surface-bound ligands can influence the response of cells, as can the mechanical compliance of a substrate material, which dictates how it will deform under forces applied by the cell. Due to differing culture conditions (time, serum concentration, confluency, etc.), the cellular status obtained can be distinct from one surface to another, and from one condition to another. Thus, special efforts to control cellular status can be necessary in order to develop biosensor-based cell assays.

Cells are dynamic objects with relatively large dimensions—typically in the range of tens of microns. Even without stimulation, cells constantly undergo micromotion—a dynamic movement and remodeling of cellular structure, as observed in tissue culture by time lapse microscopy at the sub-cellular resolution, as well as by bio-impedance measurements at the nanometer level.

Under un-stimulated conditions cells generally produce an almost net-zero DMR response as examined with a RWG biosensor. This is partly because of the low spatial resolution of optical biosensors, as determined by the large size of the laser spot and the long propagation length of the coupled light. The size of the laser spot determines the size of the area studied—and usually only one analysis point can be tracked at a time. Thus, the biosensor typically measures an averaged response of a large population of cells located at the light incident area. Although cells undergo micromotion at the single cell level, the large populations of cells give rise to an average net-zero DMR response. Furthermore, intracellular macromolecules are highly organized and spatially restricted to appropriate sites in mammalian cells. The tightly controlled localization of proteins on and within cells determines specific cell functions and responses because the localization allows cells to regulate the specificity and efficiency of proteins interacting with their proper partners and to spatially separate protein activation and deactivation mechanisms. Because of this control, under un-stimulated conditions, the local mass density of cells within the sensing volume can reach an equilibrium state, thus leading to a net-zero optical response. In order to achieve a consistent optical response, the cells examined can be cultured under conventional culture conditions for a period of time such that most of the cells have just completed a single cycle of division.

Living cells have exquisite abilities to sense and respond to exogenous signals. Cell signaling was previously thought to function via linear routes where an environmental cue would trigger a linear chain of reactions resulting in a single well-defined response. However, research has shown that cellular responses to external stimuli are much more complicated. It has become apparent that the information that cells receive can be processed and encoded into complex temporal and spatial patterns of phosphorylation and topological relocation of signaling proteins. The spatial and temporal targeting of proteins to appropriate sites can be crucial to regulating the specificity and efficiency of protein-protein interactions, thus dictating the timing and intensity of cell signaling and responses. Pivotal cellular decisions, such as cytoskeletal reorganization, cell cycle checkpoints and apoptosis, depend on the precise temporal control and relative spatial distribution of activated signal-transducers. Thus, cell signaling mediated through a cellular target such as G protein-coupled receptor (GPCR) typically proceeds in an orderly and regulated manner, and consists of a series of spatial and temporal events, many of which lead to changes in local mass density or redistribution in local cellular matters of cells. These changes or redistribution, when occurring within the sensing volume, can be followed directly in real time using optical biosensors

### DMR Signal is a Physiological Response of Living Cells

Through comparison with conventional pharmacological approaches for studying receptor biology, it has been shown that when a ligand is specific to a receptor expressed in a cell system, the ligand-induced DMR signal is receptor-specific, dose-dependent and saturate-able. For a great number of G protein-coupled receptor (GPCR) ligands, the efficacies (measured by EC₅₀ values) are found to be almost identical to those measured using conventional methods. In addition, the DMR signals exhibit expected desensitization patterns, as desensitization and re-sensitization is common to all GPCRs. Furthermore, the DMR signal also maintains the fidelity of GPCR ligands, similar to those obtained using conventional technologies. In addition, the biosensor can distinguish full agonists, partial agonists, inverse agonists, antagonists, and allosteric modulators. Taken together, these findings indicate that the DMR is capable of monitoring physiological responses of living cells.

### DMR Signals Contain Systems Cell Biology Information of Ligand-Receptor Pairs in Living Cells

The stimulation of cells with a ligand leads to a series of spatial and temporal events, non-limiting examples of which include ligand binding, receptor activation, protein recruitment, receptor internalization and recycling, second messenger alternation, cytoskeletal remodeling, gene expression, and cell adhesion changes. Because each cellular event has its own characteristics (e.g., kinetics, duration, amplitude, mass movement), and the biosensor is primarily sensitive to cellular events that involve mass redistribution within the sensing volume, these cellular events can contribute differently to the overall DMR signal. Chemical biology, cell biology and biophysical approaches can be used to elucidate the cellular mechanisms for a ligand-induced DMR signal. Recently, chemical biology, which directly uses chemicals for intervention in a specific cell signaling component, has been used to address biological questions. This is possible due to the identification of a great number of modulators that specifically control the activities of many different types of cellular targets. This approach has been adopted to map the signaling and its network interactions mediated through a receptor, including epidermal growth factor (EGF) receptor, and G_{q}- and Gₛ-coupled receptors.

EGFR belongs to the family of receptor tyrosine kinases. EGF binds to and stimulates the intrinsic protein-tyrosine kinase activity of EGFR, initiating a signal transduction cascade, principally involving the MAPK, Akt and INK pathways. Upon EGF stimulation, there are many events leading to mass redistribution in A431 cells—a cell line endogenously over-expressing EGFRs. It is known that EGFR signaling depends on cellular status. As a result, the EGF-induced DMR signals are also dependent on the cellular status. In quiescent cells obtained through 20 hr culturing in 0.1% fetal bovine serum, EGF stimulation leads to a DMR signal with three distinct and sequential phases: (i) a positive phase with increased signal (P-DMR), (ii) a transition phase, and (iii) a decay phase (N-DMR). Chemical biology and cell biology studies show that the EGF-induced DMR signal is primarily linked to the Ras/MAPK pathway, which proceeds through MEK and leads to cell detachment. Two lines of evidence indicate that the P-DMR is mainly due to the recruitment of intracellular targets to the activated receptors at the cell surface. First, blockage of either dynamin or clathrin activity has little effect on the amplitude of the P-DMR event. Dynamin and clathrin, two downstream components of EGFR activation, play crucial roles in executing EGFR internalization and signaling. Second, the blockage of MEK activity partially attenuates the P-DMR event. MEK is an important component in the MAPK pathway, which first translocates from the cytoplasm to the cell membrane, followed by internalization with the receptors, after EGF stimulation.

On the other hand, the N-DMR event is due to cell detachment and receptor internalization. Fluorescent images show that EGF stimulation leads to a significant number of receptors internalized and cell detachment. It is known that blockage of either receptor internalization or MEK activity prevents cell detachment, and receptor internalization requires both dynamin and clathrin. This indicates that blockage of either dynamin or clathrin activity should inhibit both receptor internalization and cell detachment, while blockage of MEK activity should only inhibit cell detachment, but not receptor internalization. As expected, either dynamin or clathrin inhibitors completely inhibit the EGF-induced N-DMR (˜100%), while MEK inhibitors only partially attenuate the N-DMR (˜80%). Fluorescent images also confirm that blocking the activity of dynamin, but not MEK, impairs the receptor internalization

### DMR Signals Contain Systems Cell Pharmacology Information of a Ligand Acting on Living Cells.

Since the DMR signal is an integrated cellular response consisting of contributions of many cellular events involving dynamic redistribution of cellular matters within the bottom portion of cells, a ligand-induced biosensor signal, such as a DMR signal contains systems cell pharmacology information. It is known that GPCRs often display rich behaviors in cells, and that many ligands can induce operative bias to favor specific portions of the cell machinery and exhibit pathway-biased efficacies. Thus, it is highly possibly that a ligand can have multiple efficacies, depending on how cellular events downstream of the receptor are measured and used as readout(s) for the ligand pharmacology. It is difficult in practice for conventional cell assays, which are mostly pathway-biased and assay only a single signaling event, to systematically represent the signaling potentials of GPCR ligands. However, because label-free biosensors cellular assays do not require prior knowledge of cell signaling, and are pathway-unbiased and pathway-sensitive, these biosensor cellular assays are amenable to studying ligand-selective signaling as well as systems cell pharmacology of any ligands.

### Biosensor Parameters

A label-free biosensor such as RWG biosensor or bioimpedance biosensor is able to follow in real time ligand-induced cellular response. The non-invasive and manipulation-free biosensor cellular assays do not require prior knowledge of cell signaling. The resultant biosensor signal contains high information relating to receptor signaling and ligand pharmacology. Multi-parameters can be extracted from the kinetic biosensor response of cells upon stimulation. These parameters include, but not limited to, the overall dynamics, phases, signal amplitudes, as well as kinetic parameters including the transition time from one phase to another, and the kinetics of each phase (see Fang, Y., and Ferrie, A. M. (2008) “label-free optical biosensor for ligand-directed functional selectivity acting on β2 adrenoceptor in living cells”. FEBS Lett. 582, 558-564; Fang, Y., et al., (2005) “Characteristics of dynamic mass redistribution of EGF receptor signaling in living cells measured with label free optical biosensors”. Anal. Chem., 77, 5720-5725; Fang, Y., et al., (2006) “Resonant waveguide grating biosensor for living cell sensing”. Biophys. J., 91, 1925-1940).

### EXAMPLES

### Example 1

### The Characteristics of Known hERG Activator Mallotoxin with Label-Free Biosensor Cellular Assays

Materials and Methods

### Cell Culture

All cell culture reagents were purchased from Invitrogen GIBCO cell culture products. HEK293 and HT29 cells were purchased from ATCC. HEK293 cells were maintained in MEM-GlutoMax with 10% fetal bovine serum and 1% Pennicillin/streptomycin according to ATCC's instructions. HT29 cells were maintained in McCoy's 5A medium with 10% fetal bovine serum and 1% Pennicillin/streptomycin. HEK hERG stable cell line (HEK-hERG) was maintained according to Sun et al. (J. Biol. Chem. 2006, 281, 5877). Cells were subcultured 1-2 times per week and cell passage less than 15 was used for all experiments.

### Compounds

Mallotoxin, AG-126, niflumic acid, flufenamic acid, diflunisal, dofetilide, and tyrphostin 51 were purchased from Enzo Lifesciences. Cisapride, astemizole, sertindole, fibronectin were purchased from Sigma-Aldrich. Dofetilide was purchased from Fisher Scientific.

### Label-Free Biosensor Cellular Assays

Epic® wavelength interrogation system (Corning Inc., Corning, N.Y.) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of ˜15 sec.

Cells were plated in 384-well Epic® cell culture treated plate (Corning Cat#5040) 16-20 hours before assay (15000 cells/well for HEK293 and HEK-hERG cells, 30000 cells/well for HT29 cells). For both HEK-hERG and its parental HEK293 cells, each well was coated with 10 μl 5 μg/ml fibronectin. One hour before assay, cells were washed twice on a BioTek ELx405 Select washer with Hank's Balanced Salt Solution (HBSS) containing 20 mM Hepes. Cells were incubated in 40 μl/well HBSS at 28° C. inside the Epic system for one hour. For each assay, a 2-min baseline was initiated, followed by addition of 10 μl compound solutions (5×) and the cell responses were recorded continuously for one hour.

All studies were carried out at a controlled temperature (28° C.). At least two independent sets of experiments, each with at least three replicates, were performed. The assay coefficient of variation was found to be <10%. All dose-dependent responses were analyzed using non-linear regression method with the GraphPad Prism 5.

### Rb⁺ Flux Assay

Rb⁺ flux assay was performed using HEK-hERG cells as described in previous literature (Sun, H., et al., J. Biol. Chem. 2006, 281, 5877). Briefly, 50,000 cells per well were plated in 96-well tissue culture treated plates 20 hours before assay. In the next day, cells were incubated with complete cell culture medium containing 5 mM RbCl for 3 hours at 37° C. with 5% CO₂. Then compounds (10×) diluted in HBSS were added into the cell culture medium and cells were incubated at 37° C. with 5% CO₂ for another hour. Cells were washed twice with Rb⁺ free cell culture medium and incubated with 180 μl/well of cell culture medium containing different concentration of KCl for exactly 10 minutes. The supernatant from each well was transferred to a new 96 well plate immediately. Cells were lysed with 180 μl/well 0.5% Triton-100 in HBSS. The Rb⁺ concentration of each sample was determined by ICR8000 (Aurora Biomed Inc.).

e. Automated Patch Clamp Recording Using Ionworks

CHO—K1 cells stably expressing hERG channel (CHO-hERG) were cultured in T175 flask till about 70% confluent. Cells were washed twice with PBS, then 2.5 ml 0.25% Trypsin/EDTA was mixed with 2.5 ml PBS and added to the T175 flask. Cells were incubated about 2 minutes with the diluted Trypsin/EDTA solution at 37° C., then were continuously incubated about 3 minutes at room temperature. 20 ml fresh medium were added to suspend the cells and transfer to a 50 ml tube. Cells were centrifuged down at 750 rpm for 5 minutes. The extra medium was removed and cells were resuspended in 6 ml External Buffer (137 mM NaCl, 4 mM KCl, 1.8 mM CaCl₂, 1 mM MgCl₂, 10 mM HEPES, 10 mM glucose, pH 7.4). Then cells were centrifuged again at 450 rpm for another 5 minutes. Finally cells were resuspended in 4 ml the External Buffer, cell number was counted using a hemacytometer. Cell suspension was diluted to 2.5×10⁶ cells/ml with the External Buffer. 4 ml of the resuspended cells were added to the cell reservoir in IonWorks. The Internal solution used contains: 40 mM KCl, 100 mM K-Gluconate, 3.2 mM MgCl₂, 2 mM CaCl₂, 5 mM HEPES, pH 7.25 (adjusted with KOH). 5 mg Amphotericin B from 200 ul DMSO stock was added to 65 ml the Internal solution and mixed well to achieve electrical access to the interior of cells on the patch plate.

Compounds were prepared from 10 mM DMSO stock and diluted in the External Buffer to make 3× compound solution. 600 μl/well of the 3× compound solution was transferred to a 384 well plate in Row A, B, C and D. PD11857 (Sigma-Aldrich), a reported hERG channel activator was used as activator positive control (final concentration 30 and 50 μM). hERG blocker dofetilide was used as blocker positive control (final concentration 100 nM). The final DMSO concentration in the IonWorks Quattro PatchPlate PPC plate (Cat#9000-0902, Molecular Devices) was 0.5%. Final compound concentration was 50 μM. Each compound was added to four wells of one PPC plate.

hERG currents were recorded on IonWorks Quattro (Molecular Devices). To record hERG current, the cells were clamped at −80 mV initially, then followed by a 5-s depolarization at +40 mV to activate the channels. Tail currents were measured during an ensuing return to −35 mV. Data analysis were done using IonWorks Quttro® System Software version 2.0.4.4. Data from wells with seal resistance less than 50 MΩ or hERG tail currents less than 0.1 nA were filtered out. Activator hits were selected if hERG tail currents ratio (post/pre-compound) is greater than mean+2SD (standard deviation) of the average DMSO control. Inhibitor hits were selected if hERG tail currents ratio (post/pre-compound) is less than mean-2SD of the average DMSO control.

Results

Three different types of cells: human colon cancerous cell line HT-29, HEK293 and its engineered HEK-hERG cell lines were used. HT-29 is known to endogenously express hERG channels. HEK293 is a native cell line without endogenously expressed hERG channels, while HEK-hERG is an engineered HEK293 cells having stably expressed hERG channels. Mallotoxin, a newly discovered hERG activator, was used to activate hERG channels at or near physiological conditions (i.e., 1×HBSS buffered conditions).

As shown in FIG. 1, the known hERG activator mallotoxin at 16 micromolar triggered a robust DMR signal in both HT29 and HEK-hERG cells (FIGS. 1A and B, respectively), but only triggered a net-zero DMR signal that is close to the negative control in HEK293 cells (FIG. 1C). The pretreatment of both HT29 and HEK-2hERG cells with 16 micromolar mallotoxin completely caused the desensitization of both cells to the subsequent stimulation with 16 micromolar mallotoxin (FIG. 1D). The DMR modulation index was generated by normalizing the mallotoxin DMR signal in either cell line in the presence of compound (i.e., mallotoxin) to its corresponding DMR signal in the absence of the compound, and the percentage of modulation was based on the amplitude of the mallotoxin DMR signal at a specific time (−18 min after stimulation for HEK-hERG cells, and ˜50 min after stimulation for HT29 cells, respectively). Mallotoxin is known to display polypharmacology. It is also known to be a protein kinase C (PKC) inhibitor; and PKC is a downstream signaling component of hERG activation. Thus, it would be difficult or almost impossible to assign the mode(s) of action of mallotoxin to be a hERG activator when either cell line is individually assayed with the label-free biosensor (i.e., Epic® system). Indeed, many compounds including those shown in FIGS. 2-5 also did not only lead to robust DMR signals in either HT29 or HEK-hERG cells or both, but also gave rise to certain patterns in modulating the mallotoxin responses in both cell lines. However, combining the primary DMR profiles of a compound across the three cell lines tested with the modulation index of the compound against the mallotoxin responses in both HT29 and HEK293 cells can correctly identify whether the compound is a hERG activator or not. The results shown in FIG. 1 indicate that indeed mallotoxin is a hERG activator; and the activation of hERG can result in detectable biosensor responses in the two hERG expressing cell lines.

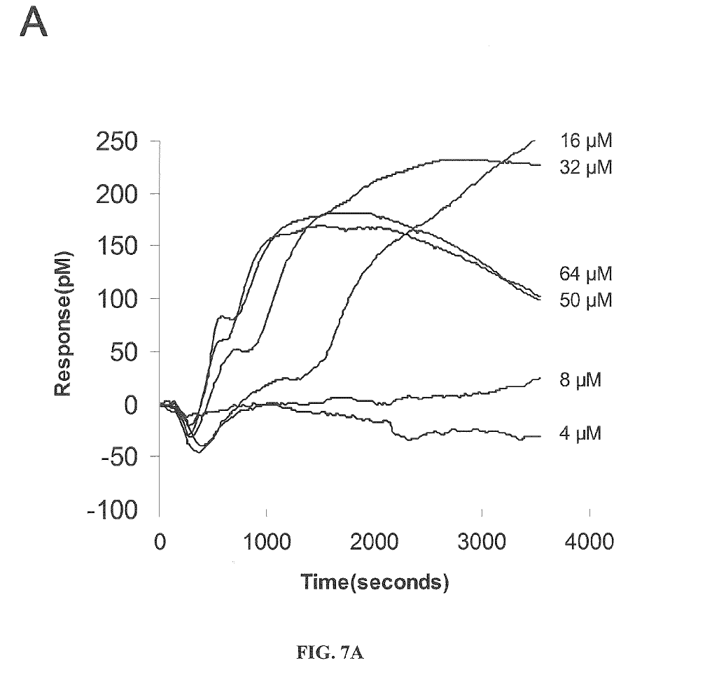

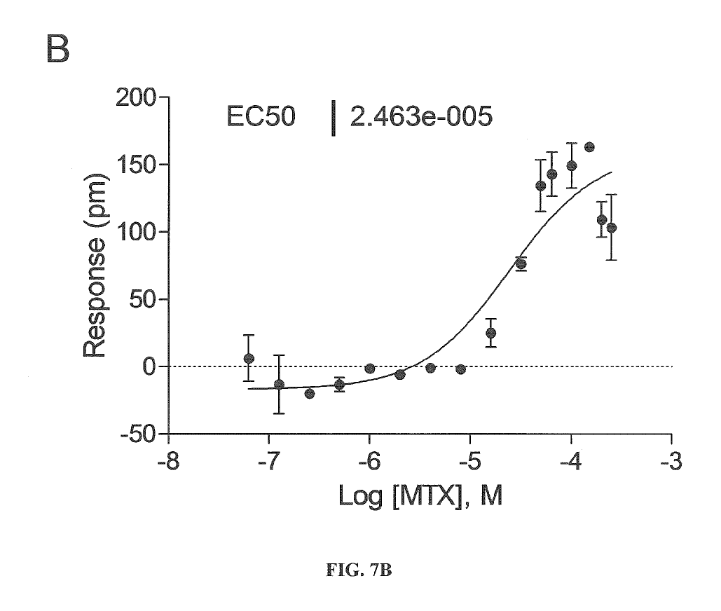

To confirm these findings, several follow-up studies were carried out. First, mallotoxin was shown to trigger a dose-dependent DMR signal in HEK-hERG cells (FIG. 7), whose amplitudes at 18 min after stimulation is saturable, leading to an apparent EC50 of ˜25 μM. Interestingly, as the doses of mallotoxin increases, its optical response becomes complicated and showed a dose-dependent switching to a complicated profile containing cellular toxicity components. Indeed, as confirmed by microscopic imaging (data not shown), mallotoxin at these high doses caused cell apoptosis (data no shown).

Second, the Rb⁺ flux assays with HEK-hERG cells confirmed that mallotoxin is a hERG activator. As shown in FIG. 6a, mallotoxin at either 10 or 50 μM led to a significant increase in Rb⁺ signaling when the cells were maintained in a buffered solution containing 5 mM KCl. Similarly, mallotoxin at 10 μM also caused an obvious increase in Rb⁺ signal when the cells were maintained in 40 mM KCl. In contrast, the known hERG blocker dofetilide caused the suppression of Rb⁺ signal in HEK-hERG cells under all three assay conditions.

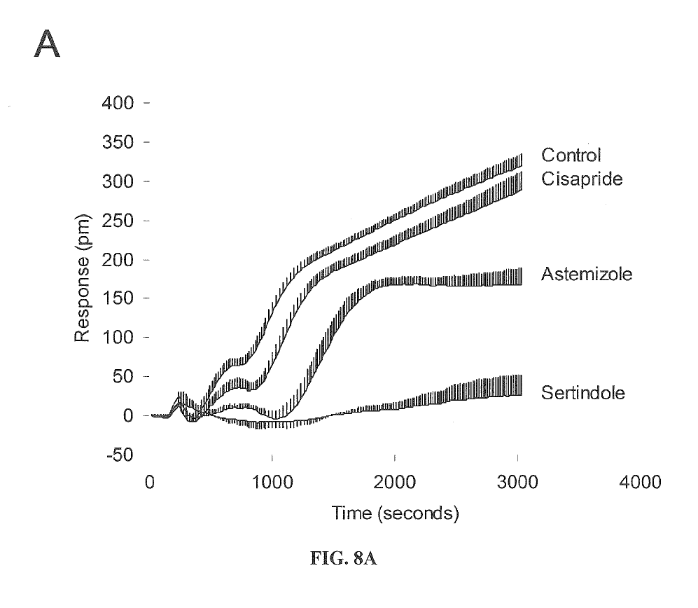

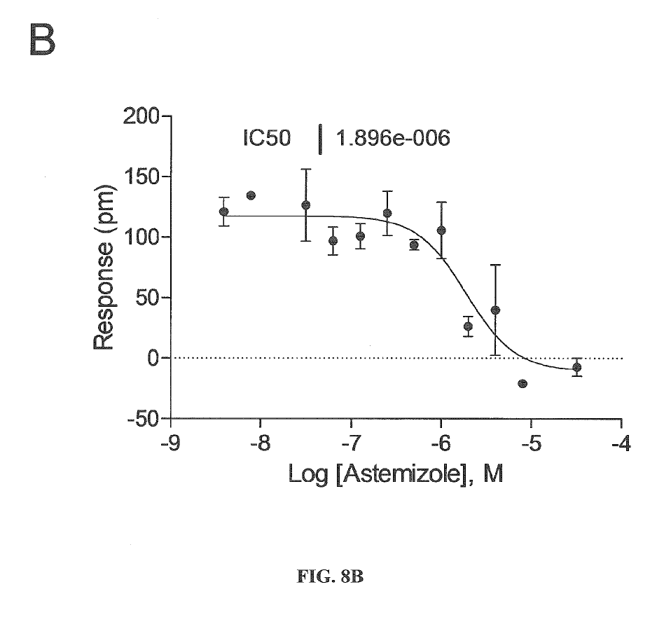

Third, the pretreatment of HEK-hERG cells individually with each of the three known HERK blockers, cisapride, astemizole, and sertindole led to a dose-dependent suppression of the mallotoxin DMR signal (FIG. 8 and data not shown). Conversely, the two large conductance potassium (BK) ion channel blockers paxilline and penitrem A showed no effect on the mallotoxin DMR signal in HEK-hERG cells (data not shown). Mallotoxin is also known to activate the BK channel. Nonetheless, these results indicate that the mallotoxin DMR signal in HEK-hERG cells is largely due to the activation of hERG channels.

Fourth, automated patch clamping recording of hERG current using CHO-hERG cells showed that mallotoxin significantly increases the hERG current, as well as hERG tail current (data not shown).

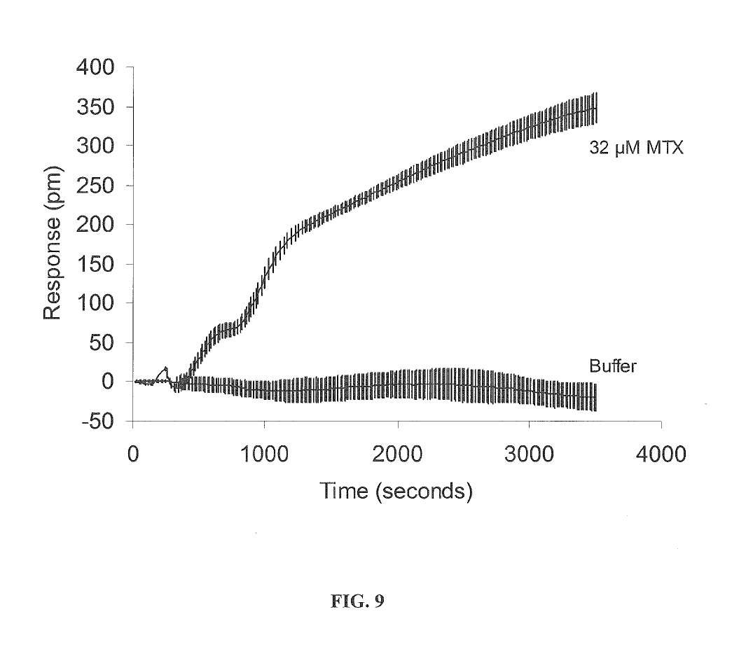

Equally important is that the mallotoxin DMR signals were extremely robust and highly reproducible (FIG. 9), showing that the present biosensor cellular assays are amenable to high throughput screening. This represents a physiological and functional HTS assay for hERG channels.

Similarly, the less potent hERG activator NS1643 also induced a detectable DMR signal in HEK-hERG cells, but not in its parent HEK-293 cells (data not shown).

### Experimental Example 2

### The Characteristics of Compounds for their Ability to Modulate the hERG Activity with Label-Free Biosensor Cellular Assays

Following the same methods disclosed, in Example 1, for both mallotoxin and NSC1643, several well-known molecules were studied for their ability to modulate the hERG activity. The non-steroidal anti-inflammatory drug flufenamic acid at 10 μM led to a robust DMR signals in both HT-29 and HEK-hERG cells (FIG. 2A and FIG. 2B, respectively), but not HEK293 cells (FIG. 2C). Flufenamic acid selectively attenuated the mallotoxin DMR signal in HT-29 cells, but not HEK-hERG cells (FIG. 2D). Although the DMR signals of flufenamic acid in both hERG-expressing cell lines are quite different from the corresponding DMR signals of mallotoxin, these results indicate that flufenamic acid acts as a weak hERG activator. Follow up studies with Rb⁺ flux assays showed that flufenamic acid indeed caused a small and dose-dependent increase in Rb⁺ signal when HEK-hERG was assayed in 5 mM KCl (FIG. 6A). Literature mining also showed that flufenamic acid (100 to 500 μM) enhanced the amplitude of outward currents evoked by depolarizing pulses in oocytes having heterologously expressed hERG channels. At potentials positive to 0 mV, an initial transient component was also evident in the presence of flufenamic acid. flufenamic acid accelerated the activation rate of hERG channels and decelerated their deactivation. Using a voltage protocol that mimicked the cardiac action potential, both flufenamic acid increased the outward current during the plateau and during the phase 3 repolarization of action potential (Malykhina, A. P., et al., European Journal of Pharmacology 2002, 452, 269-277). Consistent with these findings, our patch clamping recording led to similar results, as shown in FIG. 10B. The Curve 1030 showed the hERG currents before flufenamic acid addition, while the curve 1040 showed the hERG currents after flufenamic acid addition.

Similar results were found for niflumic acid (FIG. 10A). The Curve 1010 showed the hERG currents before niflumic acid addition, while the curve 1020 showed the hERG currents after niflumic acid addition. hERG currents were recorded on IonWorks Quattro. To record hERG current, the cells were clamped at −80 mV initially, then followed by a 5-s depolarization at +40 mV to activate the channels (phase a in FIG. 10A). Tail currents were measured during an ensuing return to −35 mV for 2 seconds (phase b in FIG. 10A). Final holding potential at −70 mV (phase c in FIG. 10B). Both flufenamic acid and niflmic acid significantly increased the tail currents (phase b). It has not been reported in literature that niflumic acid is a hERG activator.

Similarly, the prostaglandin synthetase inhibitor diflunisal at 10 μM also triggered a robust DMR signal in HT-29 (FIG. 3A), and a moderate DMR signal in HEK-hERG cells (FIG. 3B), but not HEK293 cells (FIG. 3C). The pretreatment of cells with diflunisal completely desensitized the HT29 cells responding to mallotoxin (16 μM), but not HEK-hERG cells (FIG. 3D). Diflunisal only at 50 μM triggered a detectable increase in the Rb⁺ flux signal in the 5 mM KCl maintained HEK-hERG cells (FIG. 6A). However, patch clamping recording showed that diflunisal acts as a hERG blocker, as evidenced by the suppression of the hERG tail current (FIG. 11B). These results indicate that diflunisal also acts as a weak hERG pathway activator, but a hERG current inhibitor.

On the other hand, although the ERK2 inhibitor AG-126 also triggered a DMR signal in HT29 cells similar to the corresponding DMR of diflunisal (FIG. 4A), AG-126 did not result in any obvious DMR in both HEK-hERG and HEK-293 cells (FIG. 4B and FIG. 4C, respectively). In addition, AG-126 only partially attenuated the mallotoxin DMR signal in HT-29 but not HEK-293 cells (FIG. 4D). Furthermore, AG126 has little or no impact on both hERG ion flux (FIG. 6) and hERG current (FIG. 12). These results indicate that AG-126 acts as a hERG signaling pathway modulator.

Interestingly, the potent EGFR inhibitor tyrphostin 51 led to robust but different DMR signals in all three cells tested (FIG. 5A to FIG. 5C), and only partially attenuated the mallotoxin DMR signal in HT29 cells (FIG. 5D). Tyrphostin 51 is a polypharmacological compound. Beside its inhibitory effect on EGFR, it also suppresses the activity of phosphodiesterase 4, and PKC (data not shown). Although tyrphostin 51 has little impact on hERG ion flux (FIG. 6), it significantly suppressed the hERG tail current (FIG. 11A). These results indicate that tyrphostin 51 acts as a hERG channel signaling pathway modulator, and a hERG current inhibitor.

Interestingly, the classical hERG blocker sertindole did not trigger any DMR signals in all three cell lines tested (data not shown), but attenuated the mallotoxin DMR signals in both HT29 and HEK-hERG cells in a dose dependent manner (FIG. 8). It also inhibited the Rb+ ion flux via hERG in the HEK-hERG cells, and the hERG current in CHO-hERG cells (data not shown). These results suggest that sertindole is a classical hERG blocker.

These examples highlighted that the disclosed methods, combining primary profiles of molecules across three different types of cells with the DMR modulation index of the molecules against mallotoxin acting on the two hERG expressing cells, provide high resolution assay to classify potential hERG modulators into multiple categories. These classes of hERG modulators identified include, but not limited to, hERG activator (a molecule increasing hERG current and triggering signaling via hERG, for example, mallotoxin, flufenamic acid, and niflumic acid), a hERG pathway activator (a molecule triggering signaling via hERG or hERG-associated complex, but with or without impact on hERG current or hERG ion flux, for example, diflunisal, and tyrphostin 51), a hERG pathway blocker (a molecule modulating the hERG signaling, but inhibiting hERG current or hERG ion flux, for example, A126), a hERG channel blocker (a molecule inhibiting hERG current and hERG mediated signaling, for example, sertindole, dofetilide, astemizole, cisapride).

### REFERENCES

1. WO2006108183. Fang, Y., Ferrie, A. M., Fontaine, N. M., Yuen, P. K. and Lahiri, J. “Optical biosensors and cells”
2. U.S. application Ser. No. 12/623,693. Fang, Y., Ferrie, A. M., Lahiri, J., and Tran, E. “Methods for Characterizing Molecules”, Filed Nov. 23, 2009
3. U.S. application Ser. No. 12/623,708. Fang, Y., Ferrie, A. M., Lahiri, J., and Tran, E. “Methods of creating an index”, filed Nov. 23, 2009.
4. Malykhina, A. P., et al., “Fenamate-induced enhancement of heterologously expressed hERG currents in Xenopus oocytes”. European Journal of Pharmacology 2002, 452, 269-277.
5. Sun, H., et al., “Chronic inhibition of cardiac Kir2.1 and hERG potassium channels by celastrol with dual effects on both ion conductivity and protein trafficking.” J. Biol. Chem. 2006, 281, 5877-5884.

1. A method of classifying a molecule for modulating hERG activity, comprising the steps:
a. incubating a molecule individually with at least three different types of cells consisting of a native cell endogenously expressing hERG, an engineered cell stably expressing hERG and its parental cell without expressing hERG,
b. monitoring the molecule induced cellular response on each cell type with a label-free biosensor cellular assay,

2. The method of claim 1, further comprising as steps
c) incubating a label-free biosensor hERG activator with each cell type in the presence of the molecule,
d. monitoring the label-free biosensor hERG activator induced cellular response on each cell type in the presence of the molecule,
e. generating a biosensor modulation index of the molecule against the label-free biosensor hERG activator induced biosensor signals in the two hERG expressing cell types.

3. The method of claim 2, wherein the label-free biosensor hERG activator is a hERG activator, hERG ion channel activator, or hERG pathway activator.

4. The method of claim 3, wherein the hERG activator is selected from the group consisting of mallotoxin, RPR260243, NS1643, NS3623, PD-118057, PD-307243, A-935142, flufenamic acid, niflumic acid, or diflunisal.

5. The method of claim 1, wherein the native hERG expressing cell line is selected from the group consisting of a leukemia cell line, a gastric cancer cell line, a neuroblastoma cell line, a mammary carcinoma cell line, and a human colon carcinoma cell line, a cardiovascular cell line, and a neuronal cell line.

6. The method of claim 5, wherein the native hERG expressing cell line is selected from the group consisting of cell line HL60, cell line SGC7901, cell line MGC803, cell line SH-SY5Y, cell line MCF-7, cell line HT-29 HCT8, and cell line HCT116.

7. The method of claim 1, wherein the native hERG non-expressing cell line is selected from the group consisting of a human embryonic kidney cell line or Chinese Ovary hamster cell line.

8. The method of claim 1, wherein the native hERG non-expressing cell line is selected from the group consisting of cell line HEK-293 and cell line CHO—K1.

9. The method of claim 1, wherein the hERG engineered cell line is selected from HEK-hERG or CHO-hERG.

10. The method of claim 2, wherein the biosensor modulation index of the molecule is generated by normalizing the biosensor signal of the label-free biosensor hERG activator in the presence of the molecule to the biosensor signal of the label-free biosensor hERG activator in the absence of the molecule.

11. The method of claim 1, further comprising the step of comparing the effect of the molecule to the effect of a known modulator of hERG.

12. The method of claim 11, wherein the hERG modulator is a hERG activator, a hERG ion channel activator, a hERG pathway activator, a hERG blocker, or a hERG pathway inhibitor.

13. The method of claim 9, wherein the hERG modulator is selected from the group consisting of mallotoxin, RPR260243, NS1643, NS3623, PD-118057, PD-307243, and A-935142, AG-126, flufenamic acid, diflunisal, dofetilide, and tyrphostin 51, cisapride, astemizole, or sertindole.

14. The method of claim 1, further comprising the step of determining if the molecule is a hERG modulator.

15. The method of claim 14, further comprising identifying a hERG modulator when the molecule has a biosensor index similar to a hERG modulator.

16. The method of claim 14, further comprising identifying a hERG activator when the molecule has a biosensor index similar to a known hERG activator.

17. The method of claim 14, further comprising identifying a hERG inhibitor when the molecule has a biosensor index similar to a hERG inhibitor.

18. The method of claim 1, further comprising assaying the molecule in a hERG ion flux assay test.

19. The method of claim 18, wherein the ion flux assay is a Rb⁺ ion flux assay, or a hERG current assay using electrophysiology patch clamping.

20. The method of claim 14, wherein the molecule identified as a hERG activator in the label-free biosensor assay, regardless of its action in the hERG ion flux assay, is determined to be a hERG activator.

21. (canceled)

22. (canceled)


SEQUENCE LISTING (2 sequences)

SEQ ID NO: 1 - protein, 1159 aa, Homo sapiens
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 2 - DNA, 3900 bp, Homo sapiens
ccatgggctcaggatgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagttagtggggctgcccagtgtggacacgtggctcacccagggatcaaggcgctgctgggccgctccccttggaggccctgctcaggaggccctgaccgtggaaggggagaggaactcgaaagcacagctcctcccccagcccttgggaccatcttctcctgcagtcccctgggccccagtgagaggggcaggggcagggccggcagtaggtggggcctgtggtccccccactgccctgagggcattagctggtctaactgcccggaggcacccggccctgggccttaggcacctcaaggacttttctgctatttactgctcttattgttaaggataataattaaggatcatatgaataattaatgaagatgctgatgactatgaataataaataattatcctgaggagaaaa